SEQUENCE LISTING (8 sequences)

SEQ ID NO: 1 - protein, 16 aa, Artificial Sequence
  CDR
RSSQSIVHSNGNTYLQ

SEQ ID NO: 2 - protein, 7 aa, Artificial Sequence
  CDR
KVSNRLY

SEQ ID NO: 3 - protein, 9 aa, Artificial Sequence
  CDR
FQGSHVPWT

SEQ ID NO: 4 - protein, 6 aa, Artificial Sequence
  CDR
GGYLWN

SEQ ID NO: 5 - protein, 16 aa, Artificial Sequence
  CDR
YISYDGTNNYKPSLKD

SEQ ID NO: 6 - protein, 8 aa, Artificial Sequence
  CDR
YGRVFFDY

SEQ ID NO: 7 - protein, 219 aa, Artificial Sequence
  Humanized antibody immunoglobulin chain
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 447 aa, Artificial Sequence
  Humanized antibody immunoglobulin chain
QVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2013/0287763 A1
(43) Pub. Date: Oct. 31, 2013

(54) COMBINATION THERAPY FOR TREATING CANCER COMPRISING AN IGF-1R INHIBITOR AND AN AKT INHIBITOR

(71) Applicants: Sriram Sathyanarayanan, Natick, MA (US); Christopher Winter, Needham, MA (US); Andrew Bloecher, Holliston, MA (US); Erin Valentine, East Bridgewater, MA (US)

(72) Inventors: Sriram Sathyanarayanan, Natick, MA (US); Christopher Winter, Needham, MA (US); Andrew Bloecher, Holliston, MA (US); Erin Valentine, East Bridgewater, MA (US)

(73) Assignee: MERCK SHARP & DOHME CORP., Rahway, NJ (US)

(21) Appl. No.: 13/812,249

(22) PCT Filed: Jul. 25, 2011
(86) PCT No.: PCT/US2011/045191

§ 371 Date: Jul. 17, 2013

Related U.S. Application Data
(60) Provisional application No. 61/368,374, filed on Jul. 28, 2010.

(51) Int. Cl. A61K 39/395; A61K 45/06; A61K 31/4375

(52) U.S. Cl. CPC A61K 39/39558; A61K 31/4375; A61K 45/06
USPC 424/133.1

(57) ABSTRACT

The present invention relates to a method of treating cancer by administering an IGF-1R specific antibody in combination with an anti-cancer agent exemplified by an Akt pathway inhibitor. The first and second amounts together comprise a therapeutically effective amount.

### FIELD OF THE INVENTION

The present invention relates to a method of treating cancer by administering an IGF-1R specific antibody in combination with an anti-cancer agent exemplified by an Akt pathway inhibitor. The first and second amounts together comprise a therapeutically effective amount.

### BACKGROUND OF THE INVENTION

Prominent among the class of enzymes implicated in the etiology of cancer is insulin-like growth factors (IGF), e.g., insulin-like growth factor-I and insulin-like growth factor-II each of which has been implicated in exerting mitogenic activity on various cell types such as tumor cells. IGFs are structurally similar to insulin, and have been implicated as a therapeutic tool in a variety of diseases and injuries. Insulin-like growth factor-I (IGF-I) is a 7649-dalton polypeptide with a pI of 8.4 that circulates in plasma in high concentrations and is detectable in most tissues. IGF-I stimulates cell differentiation and cell proliferation, and is required by most mammalian cell types for sustained proliferation. These cell types include, among others, human diploid fibroblasts, epithelial cells, smooth muscle cells, T lymphocytes, neural cells, myeloid cells, chondrocytes, osteoblasts and bone marrow stem cells. Each of these growth factors exerts its mitogenic effects by binding to a common receptor named the insulin-like growth factor receptor-1 (IGF 1R).

The first step in the transduction pathway leading to IGF-1-stimulated cellular proliferation or differentiation is binding of IGF-I or IGF-II (or insulin) at physiological concentrations to the IGF-I receptor. Interaction of IGFs with IGF 1R activates the receptor by triggering autophosphorylation of the receptor on tyrosine residues (Butler, et al., (1998) Comparative Biochemistry and Physiology 121:19). Once activated, IGF1R, in turn, phosphorylates intracellular targets to activate cellular signaling pathways. This receptor activation is critical for stimulation of tumor cell growth and survival. Therefore, inhibition of IGF 1R activity represents a valuable potential method to treat or prevent growth of human cancers and other proliferative diseases.

There is considerable evidence for a role for IGF-I and/or IGF-IR in the maintenance of tumor cells in vitro and in vivo. For example, individuals with “high normal” levels of IGF-I have an increased risk of common cancers compared to individuals with IGF-I levels in the “low normal” range. In addition to playing a key role in normal cell growth and development, IGF-1R signaling has also been implicated as playing a critical role in growth of tumor cells, cell transformation, and tumorigenesis. Recent data impel the conclusion that IGF-IR is expressed in a great variety of tumors and of tumor lines and the IGFs amplify the tumor growth via their attachment to IGF-IR. Indeed, the crucial discovery which has clearly demonstrated the major role played by IGF-IR in the transformation has been the demonstration that the R-cells, in which the gene coding for IGF-IR has been inactivated, are totally refractory to transformation by different agents which are usually capable of transforming the cells, such as the E5 protein of bovine papilloma virus, an overexpression of EGFR or of PDGFR, the T antigen of SV 40, activated ras or the combination of these two last factors. Other key examples supporting this hypothesis include loss of metastatic phenotype of murine carcinoma cells by treatment with antisense RNA to the IGF-1R and the in vitro inhibition of human melanoma cell motility and of human breast cancer cell growth by the addition of IGF-1R antibodies.

Potential strategies for inducing apoptosis or for inhibiting cell proliferation associated with increased IGF-I, increased IGF-II and/or increased IGF-IR receptor levels include suppressing IGF-I levels or IGF-II levels or preventing the binding of IGF-I to the IGF-IR, for example, using an anti-IGF1R antibody.

Akt signaling regulates multiple critical steps in angiogenesis. The Protein kinase B (Akt/PKB) serine/threonine protein kinases play major roles in signal transduction, cell growth, proliferation and inhibition of apoptosis. There are three structurally related homologues in humans designated as Akt1, Akt2 and Akt3. Akt/PKB is a serine/threonine kinase that has a key role in the regulation of cell survival, proliferation and growth. Hallmarks of the Akt family include an amino-terminal pleckstrin homology (PH) domain, a short α-helical linker and a carboxy-terminal kinase domain. The PH domain facilitates anchorage of Akt to membrane phospholipids produced by phosphatidylinositol 3-kinase (PI3K). PI3K is activated by growth factors, cytokines and insulin and produces phosphatidylinositol 3,4-phosphate (PIP2) and phosphatidylinositol 3,4,5-phosphate (PIP3), which interact with Akt and recruit it from the cytosol to cellular membranes. Following membrane localization, Akt is phosphorylated at Thr-308 in the kinase activation loop (T-loop) by phosphoinositide-dependent kinase 1 (PDK1), which, by virtue of its own PH domain, colocalizes with Akt at sites of PIP2/PIPS production. Phosphorylation of Akt within the carboxy-terminal tail (hydrophobic motif) at Ser-473 is required for maximal activation.

The PI3K/Akt/mTOR pathway is aberrantly active in most human cancers and contributes to cell growth, proliferation, and survival. It has also been implicated in promoting therapeutic resistance. Moreover, analysis of Akt levels in human tumors showed that Akt2 is overexpressed in a significant number of ovarian and pancreatic cancers. Similarly, Akt3 was found to be overexpressed in breast and prostate cancer cell lines. Despite the need for Akt inhibitors, few are available.

What is clear from the above recitation is that while there are many compounds in ongoing or recently completed therapeutic trials, there remains an unmet need for additional therapeutic compounds capable of treating cancer, particularly early stage and advanced hyperproliferative cellular disorders such as cancer. Besides the aim to increase the therapeutic efficacy, another purpose of combination treatment detailed herein is the potential decrease of the doses of the individual components in the resulting combinations in order to decrease unwanted or harmful side effects caused by higher doses of the individual components.

The present invention attends to the above need by providing efficacious methods for the treatment of cancer, comprising a combination treatment protocol that result in decreased side effects and is effective at treating and controlling malignancies.

### SUMMARY OF THE INVENTION

The invention provides an improved combination therapeutic and methods for the treatment of hyperproliferative diseases, such as cancer, in a mammal, typically a human, by administering a combination of an IGF-1R inhibitor, e.g., dalotuzumab, that specifically binds to human Insulin-Like Growth Factor receptor Type 1 (IGF-1R) in concert with an Akt inhibitor, e.g., MK-2206.

A surprising feature of the present invention is the discovery that the combination of a first treatment procedure that includes administration of an IGF-1R inhibitor together with a second treatment procedure comprising an Akt inhibitor is sufficient to treat various cancers that otherwise may develop resistance to treatment with an Akt inhibitor administered alone.

Antibodies and antigen-binding fragments thereof as used herein include monoclonal, polyclonal, chimeric, single chain, bispecific, and humanized or optimized antibodies as well as Fab fragments, such as those fragments which maintain the binding specificity of the antibodies to the IGF-1R proteins, including fragments thereof that express the same epitope as that bound by the antibodies of the invention, as described herein, and a second treatment procedure using one or more Akt inhibitors, as described herein, can provide therapeutically effective anticancer effects. Each of the treatments (administration of the IGF-1R inhibitor and administration of an Akt inhibitor) is used in an amount or dose that in combination with the other provides a therapeutically effective treatment. The treatment protocol need not be restricted to the first treatment procedure being limited to an IGF-1R inhibitor and the second treatment procedure being limited to the administration of an Akt inhibitor. In a separate embodiment, the cancer is a hyper-proliferative disorder which is more responsive to the combination therapeutic than to administration of each component of the therapeutic combination administered alone.

The combination therapy can act through the induction of cancer cell differentiation, cell growth arrest and/or apoptosis. Furthermore, the effect of the IGF-1R inhibitor and the anti-cancer agent, e.g., Akt inhibitor may be additive or synergistic. The combination therapy is particularly advantageous, since the dosage of each agent in a combination therapy can be reduced as compared to monotherapy with the agent, while still achieving an overall anti-tumor effect.

Indeed a broad aspect of the invention relies on the surprising discovery of a method of treating cancer in a subject in need thereof, by administering to a subject in need thereof a first amount of an IGF-1R inhibitor, preferably an IGF-1R antibody in a first treatment procedure, and a second amount of an anti-cancer agent, e.g., Akt inhibitor or a pharmaceutically acceptable salt or hydrate thereof, in a second treatment procedure, wherein the first and second treatments together comprise a therapeutically effective amount.

Treatment of cancer, as used herein, refers to partially or totally inhibiting, delaying or reversing the progression of cancer including cancer metastasis; inhibiting, delaying or reversing the recurrence of cancer including cancer metastasis; in a mammal, for example a human.

The methods of the present invention are useful in the treatment in a wide variety of cancers, including but not limited to solid tumors (e.g., tumors of the lung, breast, colon, prostate, bladder, rectum, brain or endometrium), hematological malignancies (e.g., leukemias, lymphomas, myelomas), carcinomas (e.g. bladder carcinoma, renal carcinoma, breast carcinoma, colorectal carcinoma), neuroblastoma, or melanoma. Non-limiting examples of these cancers include cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), acute lymphocytic leukemia, acute nonlymphocytic leukemia, chronic lymphocytic leukemia, chronic myelogenous leukemia, Hodgkin's disease, non-Hodgkin's lymphoma, multiple myeloma, mesothelioma, childhood solid tumors such as brain neuroblastoma, retinoblastoma, Wilms' tumor, bone cancer and soft-tissue sarcomas, common solid tumors of adults such as head and neck cancers (e.g., oral, laryngeal and esophageal), genito urinary cancers (e.g., prostate, bladder, renal, uterine, ovarian, testicular, rectal and colon), lung cancer, breast cancer, pancreatic cancer, melanoma and other skin cancers, stomach cancer, brain cancer, liver cancer, adrenal cancer, kidney cancer, thyroid cancer, basal cell carcinoma, squamous cell carcinoma of both ulcerating and papillary type, metastatic skin carcinoma, medullary carcinoma, osteosarcoma, Ewing's sarcoma, veticulum cell sarcoma, Kaposi's sarcoma, neuroblastoma and retinoblastoma.

The invention further relates to pharmaceutical combinations useful for the treatment of cancer. The pharmaceutical combination comprises a first amount of an IGF-1R inhibitor, e.g., dalotuzumab and second amount of an Akt inhibitor, e.g., MK-2206. The first and second amounts together comprise a therapeutically effective amount.

The invention further relates to the use of a first amount of an IGF-1R inhibitor and a second amount of an Akt inhibitor for the manufacture of a medicament for treating cancer.

In particular embodiments of this invention, the combination of the IGF-1R inhibitor and Akt inhibitor is additive, i.e. the combination treatment regimen produces a result that is the additive effect of each constituent when it is administered alone. In accordance with this embodiment, the amount of IGF-1R inhibitor and the amount of the Akt inhibitor together constitute an effective amount to treat cancer.

In another particular embodiment of this invention, the combination of the IGF-1R inhibitor and Akt inhibitor is considered therapeutically synergistic when the combination treatment regimen produces a significantly better anticancer result (e.g., cell growth arrest, apoptosis, induction of differentiation, cell death) than the additive effects of each constituent when it is administered alone at a therapeutic dose. Standard statistical analysis can be employed to determine when the results are significantly better. For example, a Mann-Whitney Test or some other generally accepted statistical analysis can be employed.

In one aspect, the method of the invention comprises administering to a patient in need thereof a first amount of an IGF-1R antibody, comprising light and heavy chains as described infra, in a first treatment procedure, and a second amount of an anti-cancer agent, preferably an Akt inhibitor, e.g., MK-2206, in a second treatment procedure. The first and second treatments together comprise a therapeutically effective amount.

In another aspect of the invention, the IGF-1R antibody is dalotuzumab (MK-0646).

In yet a further aspect of the invention, administration of the combination results in enhanced therapeutic efficacy relative to administration of the IGF-1R inhibitor alone.

In yet a further aspect of the invention, administration of the combination results in enhanced therapeutic efficacy relative to administration of the Akt inhibitor alone.

In another particular embodiment of this invention, the combination of the IGF-1R antibody and the Akt inhibitor is considered therapeutically synergistic when the combination treatment regimen produces a significantly better anti-cancer result (e.g., reduction in tumor volume, cell growth arrest, apoptosis, induction of differentiation, cell death etc.) than the additive effects of each constituent when it is administered alone at a therapeutic dose. Standard statistical analysis can be employed to determine when the results are significantly better. For example, a Mann-Whitney Test or some other generally accepted statistical analysis can be employed.

The treatment procedures can take place sequentially in any order, simultaneously or a combination thereof. For example, the first treatment procedure, administration of an IGF-1R inhibitor, can take place prior to the second treatment procedure, i.e. Akt inhibitor, after the second treatment with the Akt inhibitor, at the same time as the second treatment with the Akt inhibitor, or a combination thereof. For example, a total treatment period can be decided for the IGF-1R inhibitor. The Akt inhibitor agent can be administered prior to onset of treatment with the IGF-1R inhibitor or following treatment with the IGF-1R inhibitor. In addition, treatment with the Akt inhibitor can be administered during the period of IGF-1R inhibitor administration but does not need to occur over the entire IGF-1R inhibitor treatment period. Similarly, treatment with the IGF-1R inhibitor can be administered during the period of Akt inhibitor administration but does not need to occur over the entire Akt inhibitor treatment period. In another embodiment, the treatment regimen includes pre-treatment with one agent, either the IGF-1R inhibitor or the Akt inhibitor, followed by the addition of the second agent for the duration of the treatment period.

In one particular embodiment of the present invention, the Akt inhibitor can be administered in combination with any one or more of an additional Akt inhibitor, as described herein or any combination thereof.

In another embodiment of the present invention, the IGF-1R is an IGF-1R antibody, preferably dalotuzumab or one having the light and heavy chains as set forth herein, which can be administered in combination with any one or more of another IGF-1R inhibitor, an anti-angiogenic agent, a differentiation inducing agent, a cell growth arrest inducing agent, an apoptosis inducing agent, a cytotoxic agent, or any combination thereof.

The anti-IGF-1R antibody may be administered via parenteral, e.g., subcutaneous, intratumoral, intravenous, intradermal, oral, transmucosal, or rectal administration. While not intending to be bound to a particular theory of operation, it is believed that blockade of IGF-1R mediated signaling cascade through the administration of an anti-IGF-1R antibody potentiates anti-tumor immunity by negatively modulating the signaling cascade attendant the binding of a native IGF-1R ligand to the receptor. IGF-1R inhibitors suitable for use in the present invention, include but are not limited to the IGF-1R antibody described and claimed in U.S. Pat. No. 7,241,444.

The Akt pathway inhibitor compounds of the instant invention are useful in the inhibition of the activity of the serine/threonine kinase Akt. In one particular embedment, the Akt inhibitor is MK-2206. Synthesis of MK-2206 and methods of use thereof are detailed in WO 2008/070016, Ser. No. 11/999,234 and U.S. Pat. No. 7,576,209, the entire content of each of which is incorporated by reference herein in its entirety.

Described here below is one schematic of synthesizing MK-2206. Other schemes of synthesizing MK-2206 may also be employed.

Synthesis of MK-2206 is also found in WO2008/070016.

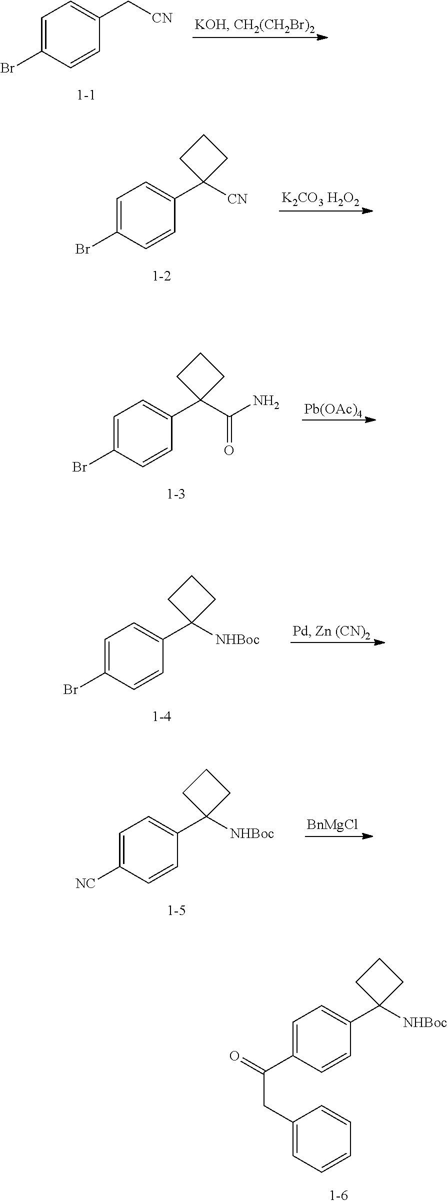

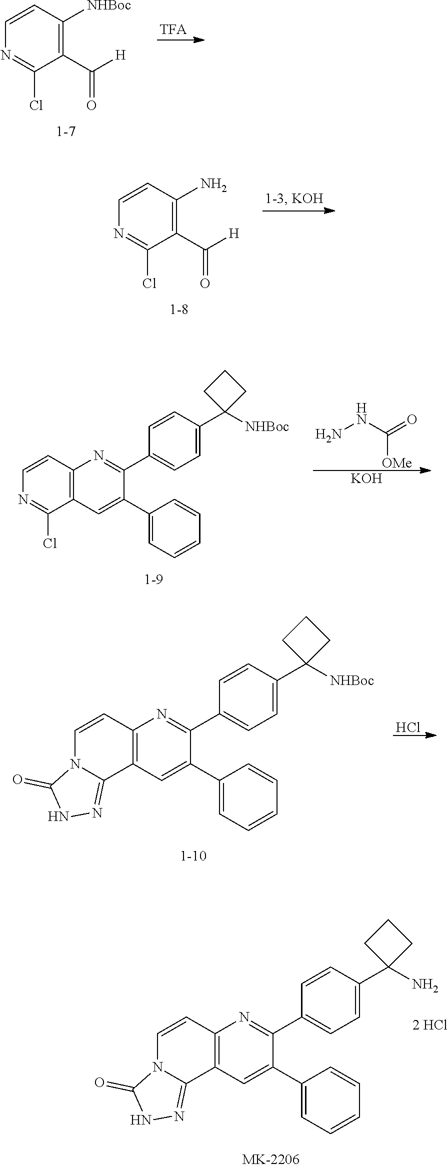

### 1-(4-bromophenyl)cyclobutanecarbonitrile (1-2)

TBAB (1.61 g, 0.5 mmol), dibromopropane (22.2 g, 110 mmol), and nitrile 1-1 (19.6 g, 100 mmol) were added to a stirred solution of KOH (31.17 g, 500 mmol) in a mixture of 15 mL of water and 200 mL of toluene (temperature maintained between 72 and 79° C. The mixture was heated by steam and was stirred at 99-108° C. for 2.5 h. The mixture was cooled to 80 0° C. and 200 mL of heptane was added. After the resulting mixture was cooled to RT with stirring, the top clear solution was filtered, washed with water (3×30 mL) and concentrated in vacuo to give oily product 1-2.

### 1-(4-bromophenyl)cyclobutanecarboxamide (1-3)

H2O2 (30% 11.3 mL, 118 mmol) was added over 3 h to a stirred mixture of nitrile 1-2 (13.88 g, ˜58.9 mmol) and K2CO3 (1.62 g, 11.8 mol) in 59 mL of DMSO at 40-87° C., cooling with a water bath. The resulting mixture was cooled to 27° C. and water (100 mL) was added over 30 min. Crystalline product 1-3 formed. More water (100 mL) was added over 1 h. The resulting slurry was aged at RT for 16 h before filtration. The cake was rinsed with 100 mL of water and then with 100 mL of heptane. After drying in a vacuum oven at 50° C., product 1-3 was obtained as a white solid.

### tert-butyl[1-(4-bromophenyl)cyclobutyl]carbamate (1-4)

Pb(OAc)4 (25.7 g, 25.7 mmol) was added to a stirred solution of amide 1-3 (12.7 g, 50 mmol) in 64 mL of t-BuOH at 57° C. to 86° C. cooling with a water bath. The resulting mixture was stirred at 65-86° C. for 0.5 h. The mixture was cooled to 26° C. and 12.7 g of Na2CO3 were added followed by 65 mL MTBE. After 10 min, the mixture was filtered. The cake was rinsed with 10 L of MTBE and the combined filtrate was washed with 20 mL of water and the organic layer was then washed with 3×10 mL of 10% KHCO3 (caution: bubbling) dried over Na2SO4 and concentrated in vacuo. The resulting solid was rinsed with 8 mL of IPAc and 8 mL of heptane and dried in a vacuum oven at 40° C. to give product 1-4 as a grey solid.

### tert-butyl[1-(4-cyanophenyl)cyclobutyl]carbamate (1-5)

A stirred slurry of Pd2dba3 (101 mg; 1 mol %) and dppf (122 mg; 2 mole %) in DMF (25 mL) was sparged with nitrogen for 5 min and then warmed to 65° C. and aged for 30 min. At this temp was added the aryl bromide 1-4 (3.6 g, 11 mmol), zinc powder (51 mg; 6 mol %) and the zinc cyanide (777 mg; 0.60 equiv) rinsing with DMF (5 mL). The solution was heated to 92-95° C. and aged for 4 h. The solution was cooled to RT overnight and filtered through a pad of Solka Floc, rinsing the cake with DMF (5 mL). Water (30 mL) was added over 3.5 h at 25-33° C., along with seed. After aging overnight at RT, the resulting crystalline solution was filtered and washed with aqueous methanol and dried overnight to yield 1-5 as a yellow solid.

### tert-butyl ({[4-phenylacetyl)phenyl]cyclobutyl}carbamate (1-6)

Benzyl Grignard (19 mL, 38.5 mmol) was added to a stirred, slightly cloudy solution of nitrile 1-5 (3 g, 11 mmol) in THF (25 mL) cooled to ca. −20° C. at a rate such that the reaction temperature did not warm above −10° C. The solution was aged for 3-4 hours keeping the reaction temperature between −10° C. and −20° C. The stirred solution was cooled to −30° C. and added to a 15 wt % aqueous citric acid solution (60 mL) which was previously cooled to 5-10° C., maintaining the temperature below 15° C. The layers were separated and the aqueous layer was washed with MTBE.

The organic layers were combined, washed with half saturated brine (60 mL), and concentrated under reduced pressure. Heptane was added and the mixture was concentrated to a slurry which was filtered, washed with heptane (15 mL) and dried under nitrogen to give 1-6.

### 4-Amino-2-chloronicotinaldehyde (1-8)

Trifluoroacetic acid (17.4 mL, 234 mmol) was added carefully to a stirred mixture of Boc aldehyde 1-7 (20 g, 78.1 mmol) and dichloromethane (60 mL) keeping the temperature below 25° C. The solution was warmed to 35° C., aged overnight (vigorous off-gassing) and then cooled to room temperature. 25 mL of MTBE was added and the resulting white slurry was aged for one hour, filtered, and the filter cake rinsed with MTBE (10 mL×2). Solid 1-8 TFA salt was dried under vacuum.

### tert-butyl {1-[4-(5-chloro-3-phenyl-1,6-naphthyridin-2-yl)phenyl]cyclobutyl}carbamate (1-9)

45 wt % Potassium hydroxide solution (18 mL; 5 equiv) was added dropwise over 20 minutes to a stirred mixture of chloropyridine TFA salt 1-8 (19.5 g), cyclobutylamino ketone 1-6 (26 g) and isopropanol (200 mL) keeping the temperature below 24° C. After 1 h, water (100 mL) was added and after a further 1 h the resulting slurry was filtered, washing with 2:1 IPA/water (30 mL, then 24 mL) then with water (80 mL then 2×60 mL). The solid was dried under nitrogen flow to afford 1-9 as an off-white solid.

### tert-butyl {1-[4-(3-oxo-9-phenyl-2,3-dihydro[1,2,4]triazolo[3,4-f][1,6]naphthyridin-8-yl)phenyl]cyclobutyl}carbamate (1-10)

A stirred slurry of chloronapthyridine 1-9 (1.8 g), methyl hydrazine carboxylate (0.318 g) and isopropanol (20 L) is warmed to 66° C. before becoming homogeneous. 5-6 N HCl in IPA (0.05 ml) is added and the temperature is increased to 70° C. for 16 hours and then is cooled to RT. After cooling to RT, 45 wt % potassium hydroxide solution (0.52 mL) is mixed with water (5.5 mL) and added over 15 minutes. After 30 minutes, aqueous acetic acid (0.7 mL in 6 mL water) is added followed by water (2 mL). The resulting slurry is aged at RT for three hours, filtered and washed with 1:1 IPA/water (2×2.4 mL). The product is dried under nitrogen flow then slurried in methylene chloride at 20° C. for 4 hours, filtered and dried under nitrogen flow to afford 1-10 as an off-white solid.

### 8-[4-(1-Aminocyclobutyl)phenyl]-9-phenyl[1,2,4]triazolo[3,4-f]-1,6-naphthyridin-3(2H)-one (MK-2206)

A solution of aqueous concentrated HCl (12.1 M, 1.64 mL) in ethanol (2.0 mL) was added dropwise over 30 min to a stirred slurry of 1-10 (500 mg, 0.985 mol) in ethanol (1.7 mL) and water (0.2 mL) at 50° C. After 3 hours following acid addition, the mixture was seeded and aged overnight at 50° C., cooled to room temperature and filtered. Acetyl chloride (0.5 g, 7 mmol) was added over 1 h to ethanol (2 mL) at 0° C. The solution was then cooled to room temperature and aged for 30 minutes. The filter cake was washed with this solution (1 mL×2), then with ethyl acetate (4 mL×2) and dried, finally in a vacuum oven at 75.0° C. with nitrogen sweep (50 torr) to afford MK-2206 as the bis-HCl salt.

A mono-HCl version of MK-2206 was also produced via dissolution in water. After 6 hours, the aqueous slurry turns light yellow and is filtered. Silver chloride titration of this solid reveals the presence of one equivalent of chloride.

Pharmaceutical Preparations Comprising MK-2206

Preparations of the monohydrochloride salt of MK-2206 may be prepared as a tablet involving roller compression granulation followed by milling, mixing with the other inactive ingredients, compression, and film coating.

Some of the diluents or fillers for use in this formulation are preferably swellable agents, and may include, but are not limited to, various grades of microcrystalline cellulose, such as Avicel PH101, Avicel PH102, & Avicel PH200. If microcrystalline cellulose is added, it is preferably from about 50 to 180 microns in size, more preferably about 100. Avicel PH 101 has a mean particle size of about 50; Avicel PH 102 has a mean particle size of about 100; and Avicel PH 200 has a mean particle size of about 190 microns. Preferably, the preferred microcrystalline cellulose is Avicel PH 102.

The edible calcium salts suitable for use herein include but are not limited to, dibasic calcium phosphate dihydrate, calcium phosphate anhydrous, and tribasic calcium phosphate; or mixtures thereof. A preferred edible calcium salt is the dibasic calcium phosphate anhydrous, which also provides good compressibility.

Suitable ratios for particular diluents however, are described below: For microcrystalline cellulose:Dibasic calcium phosphate, dihydrate, from about 2 to about 4:1, preferably from about 2.6-3.1:1; For microcrystalline cellulose:Calcium phosphate, anhydrous from about 1 to about 3:1, preferably from about 1.6:1, microcrystalline cellulose:Tribasic calcium phosphate, from about 2 to about 4:1, preferably from about 3.1:1.

A preferred disintegrating agent is sodium croscarmellose. Preferably, the sodium croscarmellose is present in an amount of about 2 to about 5% w/w.

A preferred lubricant is magnesium stearate.

An aspect of the present invention is a process for preparing a tablet formulation which comprises:

a) blending together to form an intragranular mixture of the active monohydrochloride salt of MK-2206, microcrystalline cellulose, an edible calcium salt, disintegrant, and lubricant;

b) roller compression granulation of the mixture of step (a) for the purpose of preparing granules;

c) lubricating the granulation from step (b);

d) compacting the lubricated granulates of step (c) into concave tablet; and

e) film coating tablets from step (d).

Biologic agents suitable for use in the present invention include, but are not limited to immuno-modulating proteins, monoclonal antibodies against tumor antigens, tumor suppressor genes, and cancer vaccines. For example, the immuno-modulating protein can be interleukin 2, interleukin 4, interleukin 12, interferon E1 interferon D, interferon alpha, erythropoietin, granulocyte-CSF, granulocyte, macrophage-CSF, bacillus Cahnette-Guerin, levamisole, or octreotide. Furthermore, the tumor suppressor gene can be DPC-4, NF-1, NF-2, RB, p53, WT1, BRCA, or BRCA2.

The IGF-1R inhibitor (e.g., dalotuzumab), and the anti-cancer agent, e.g., Akt inhibitor can be administered by any administration method known to a person skilled in the art. Examples of routes of administration include but are not limited to oral, parenteral, intraperitoneal, intravenous, intraarterial, transdermal, sublingual, intramuscular, rectal, transbuccal, intranasal, liposomal, via inhalation, vaginal, intraoccular, via local delivery by catheter or stent, subcutaneous, intraadiposal, intraarticular, intrathecal, or in a slow release dosage form.

The IGF-1R inhibitor or any one of the Akt inhibitors can be administered in accordance with any dose and dosing schedule that, together with the effect of the Akt inhibitor, achieves a dose effective to treat cancer. However, a preferred dose of for the Akt inhibitor is 60 mg once every other day and/or 200 mg once a week.

A preferred embodiment of the invention is drawn to a method of treating cancer in a subject in need thereof, by administering to a subject in need thereof a first amount of an IGF-1R inhibitor in a first treatment procedure, and a second amount of an Akt inhibitor or a pharmaceutically acceptable salt or hydrate thereof at a total daily dose of up to 60 mg once every other day (QOD) or 200 mg once a week in a second treatment procedure, wherein the first and second amounts together comprise a therapeutically effective amount.

Another preferred embodiment of the invention is drawn to a method of treating cancer in a subject in need thereof, by administering to a subject in need thereof a first amount of an IGF-1R inhibitor at a total dose of 20 mg/kg once weekly in a first treatment procedure, and a second amount of an Akt inhibitor or a pharmaceutically acceptable salt or hydrate thereof at a total daily dose of up to 60 mg once every other day (QOD) in a second treatment procedure, wherein the first and second amounts together comprise a therapeutically effective amount. In an alternative embodiment, the IGF-1R inhibitor e.g., dalotuzumab is administered at a total dose of 10 mg/Kg once weekly. In an alternative embodiment, the Akt inhibitor (MK-2206) is administered at a total dose of 200 mg once a week.

When a composition according to this invention is administered into a human subject, the daily dosage will normally be determined by the prescribing physician with the dosage generally varying according to the age, weight, and response of the individual patient, as well as the severity of the patient's symptoms.

In one embodiment, the Akt inhibitor, e.g. MK-2206 is administered in a pharmaceutical composition, preferably suited for oral administration. In another embodiment, MK-2206 is administered orally in a gelating capsule, which can comprise excipients such as microcrystalline cellulose, croscarmellose sodium and magnesium stearate.

It is apparent to a person skilled in the art that any one or more of the specific dosages and dosage schedules of the IGF-1R inhibitors is also applicable to any one or more of the anti-cancer agents to be used in the combination treatment. Moreover, the specific dosage and dosage schedule of the anti-cancer agent can further vary, and the optimal dose, dosing schedule and route of administration will be determined based upon the specific anti-cancer agent that is being used.

The present invention also provides methods for selectively inducing terminal differentiation, cell growth arrest and/or apoptosis of neoplastic cells, thereby inhibiting proliferation of such cells in a subject by administering to the subject a first amount of an IGF-IR inhibitor or an Akt inhibitor or a pharmaceutically acceptable salt or hydrate thereof, in a first treatment procedure, and a second amount of an anti-cancer agent e.g., an Akt inhibitor or an IGF-1R inhibitor in a second treatment procedure, wherein the first and second amounts together comprise an amount effective to induce terminal differentiation, cell growth arrest of apoptosis of the cells.

The present invention also provides in-vitro methods for selectively inducing terminal differentiation, cell growth arrest and/or apoptosis of neoplastic cells, thereby inhibiting proliferation of such cells, by contacting the cells with a first amount of an IGF-1R inhibitor and a second amount of an anti-cancer agent e.g., Akt inhibitor or a pharmaceutically acceptable salt or hydrate thereof, wherein the first and second amounts together comprise an amount effective to induce terminal differentiation, cell growth arrest of apoptosis of the cells.

The combination therapy can provide a therapeutic advantage in view of the differential toxicity associated with the two treatment modalities. For example, treatment with IGF-1R inhibitors can lead to a particular toxicity that is not seen with the Akt inhibitor, and vice versa. As such, this differential toxicity can permit each treatment to be administered at a dose at which said toxicities do not exist or are minimal, such that together the combination therapy provides a therapeutic dose while avoiding the toxicities of each of the constituents of the combination agents. The same holds true for the Akt inhibitor.

Furthermore, when the therapeutic effects achieved as a result of the combination treatment are enhanced or synergistic, for example, significantly better than additive therapeutic effects, the doses of each of the agents can be reduced even further, thus lowering the associated toxicities to an even greater extent.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIGS. 1A and B detail upregulation of RTk's attendant Akt inhibition. 1A.) HT-29 cells were treated with increasing doses of MK-2206 (0.041-1.11 um) and western blotted for levels of phospho and total Akt and total IGF1R. 1B.) HT-29 cells were treated with 1 uM MK-2206 for 18 hrs and then assessed for the RTK phosphorylation state using RTK arrays. 1 denotes phospho-IGF-1R, 2 denotes phospho-IR), and 3 denotes phospho-cMET. All phospho-RTKs are measured in duplicate spots.

FIGS. 2 A-C demonstrate that AKT inhibition results in an upregulation of diverse RTKs in multiple cancer types. 2A.) SKCO1 (CRC), 2B.) HCC70 (breast cancer), and 2C.) H1793(NSCLC) cells were treated with MK-2206 (370 nM, 370 nM, 1 uM respectively) for 18 hrs and then assessed for the RTK phosphorylation state using RTK arrays. 1 phospho-IGF-1R, 2 phospho-cMET, 3 phospho-Her2, 4 phospho-Her3, 5 phospho-EphA2, 6 phospho-IR. All phospho-RTKs are measured in duplicate spots.

FIG. 3 A-D details the ability of MK-0646 (dalotuzumab) to prevent MK-2206 mediated increase in phospho-RTKs and its efficacy in combination in vitro. Figures A & C—HT-29 and SW-837 cells were treated with 123 nM MK-2206, 20.95 ug/ml MK-0646, or the combination for 18 hrs and then assessed for the RTK phosphorylation state using RTK arrays. 1 phospho-IGF-1R, 2 phospho-IR, 3 phospho-cMET. All phospho-RTKs are measured in duplicate spots. Figures B & D—HT-29 and SW-837 cells were grown in soft agar in presence of increasing amounts of MK-2206, MK-0646 at a fixed dose, or the combination and scored for colony area. Data is shown as the % inhibition relative to vehicle treated cells.

FIG. 4 shows inhibition of SKCO1 (KRASmut) tumor growth by MK-0646 & MK-2206 combination. Relative tumor volumes (n=4/group) are plotted against the duration (days) after treatment initiation. The error bars represent standard error from mean. MK-0646 and MK-2206 single agent treatments significantly inhibited tumor growth as compared to vehicle-treated animals (P<0.01; two-way ANOVA). According to the data, the combination of MK-0646 and MK-2206 significantly inhibited SKCO-1 tumor growth as compared to single agent treatments (P<0.05) or vehicle control group (P<0.001).

FIG. 5 summarizes the efficacy of a treatment protocol relative to a control, MK-0646, MK-2206 and a combination of MK-0646 & MK-2206 (MK0646/MK-2206 in two KRASmut and one BRAFmut colorectal cancer xenograft models: SKCO1 (KRASG12V), SW837 (KRASG12C) and HT29 (BRAFV600E). The MK-0646/MK-2206 combination resulted in tumor regression in one out of the three CRC models tested. However, the MK-0646/MK-2206 combination resulted in significant tumor growth inhibition, compared to standard of care (cetuximab) in all three KRAS/BRAF mutant CRC xenograft models. Bars represent mean percent tumor volume, normalized to the appropriate control groups. Error bars represent SEM. All data represent final tumor volumes at the end of 22-28 day treatment periods. Statistically significant differences are shown comparing MK-0646/MK-2206 combination groups to cetuximab or single agents. Student's t-test comparisons were used.

FIG. 6 A-C details the acute changes in IGF-1R signaling pathway following MK-0646 & MK-2206 treatment. Samples are collected 24 hrs following single dose of MK-0646 or 6 hrs following MK-2206 treatment. A and B) Total IGF-1R protein expression as assessed by IHC. Panel A represents an example of IGF-1R expression levels within tumors treated with vehicle or MK-2206. Panel B shows a representative field of view observed within tumors treated with MK-0646 or the MK-0646/MK-2206 combination. C) Tumor samples were assessed for IGF-1R pathway activity using western blot analysis, probed with indicated total or phosphor-specific antibodies.

### DETAILED DESCRIPTION OF THE INVENTION

The present invention relates to a method of treating cancer in a subject in need thereof, by administering to a subject in need thereof a first amount of an IGF-1R inhibitor or a pharmaceutically acceptable salt or hydrate thereof, in a first treatment procedure, and a second amount of an Akt inhibitor in a second treatment procedure, wherein the first and second amounts together comprise a therapeutically effective amount. The effect of the IGF-1R inhibitor and the Akt inhibitor may be additive or synergistic.

The present invention also relates to a method of treating cancer in a subject in need thereof, by administering to a subject in need thereof a first amount of dalotuzumab (MK-0646) in a first treatment procedure, and a second amount of Akt inhibitor, MK-2206 or a pharmaceutically acceptable salt or hydrate thereof in a second treatment procedure, wherein the first and second amounts together comprise a therapeutically effective amount. The effect of dalotuzumab and MK-2206 agent may be additive or synergistic.

Compositions or combinations or the present invention include an IGF1R inhibitor such as an anti-IGF1R antibody or antigen-binding fragment thereof “in association with” an AKT inhibitor; these combinations may optionally be in association with one or more further therapeutic agents or therapeutic procedures. The term “in association with” indicates that the components (e.g., dalotuzumab and MK-2206) can be formulated into a single composition for simultaneous delivery or formulated separately into two or more compositions (e.g., a kit). Furthermore, each component can be administered to a subject at a different time than when the other component is administered; for example, each administration may be given non-simultaneously (e.g., separately or sequentially) at several intervals over a given period of time. Moreover, the separate components may be administered to a patient by the same or by a different route (e.g., parenterally and orally). The compositions and combinations of the present invention include any product comprising the specified ingredients e.g., in the specified amounts, including any such product which results, directly or indirectly, from combination of the specified ingredients in the specified amounts.

The term “administration” and variants thereof (e.g., “administering” a compound) in reference to the combinations of the present invention means introducing the components into the system of a mammal or animal such as a human in need of such treatment. When the combinations disclosed herein are provided in association with one or more other therapeutic agents (e.g., a cytotoxic agent, etc.), “administration” and its variants are each understood to include concurrent and sequential introduction of the combination and the further therapeutic agents.

The term “treating” in its various grammatical forms in relation to the present invention refers to curing, reversing, attenuating, alleviating, minimizing, suppressing or halting the deleterious effects of a disease state, disease progression, disease causative agent (e.g., bacteria or viruses) or other abnormal condition. Treatment of cancer, as used herein, refers to partially or totally inhibiting, delaying or reversing the progression of cancer including cancer metastasis and primary tumor cell growth or survival, for example, in a mammal such as a human. For example, in an embodiment of the invention, one or more tumors in a patient with cancer treated with a combination of the present invention (e.g., dalotuzumab and MK-2206) shrinks in size (e.g, in volume, surface area or diameter).

In addition, the present invention includes chemoprevention of a hyperproliferative disorder such as cancer in a human.

As used herein, the term “therapeutically effective amount” is intended to qualify the combined amount of the IGF1R inhibitor (e.g., dalotuzumab) and the AKT inhibitor (e.g., MK-2206) in the combination therapy of the present invention. The combined amount will achieve the desired biological response. In the present invention, the desired biological response is partial or total inhibition, delay or reversal of the progression of primary tumors or cancer metastasis; in a mammal such as a human.

Tumor treatment is, in an embodiment of the invention, assessed by use of imaging techniques such as X-ray, ultrasound scan, MRI (magnetic resonance imaging), CAT scan or PET (positron emission tomography; e.g., fluorodeoxyglucose-PET) scan to determine if the treatment results in tumor shrinkage.

As used herein, the terms “combination treatment”, “combination therapy”, “combined treatment” or “combinatorial treatment”, “combination therapeutic” may be used interchangeably, and refer to a treatment of an individual with at least two different therapeutic agents: a first therapeutic agent, such as dalotuzumab or another IGF-1R inhibitor in association with a second therapeutic agent such as MK-2206 or some other Akt pathway inhibitor. A combinatorial treatment may include or exclude any further therapeutic agent.

The invention further relates to pharmaceutical combinations useful for the treatment of cancer. The pharmaceutical combination comprises a first amount of an IGF-1R inhibitor, e.g., dalotuzumab and a second amount of an anti-cancer agent, e.g., an Akt pathway inhibitor. The first and second amounts together comprise a therapeutically effective amount.

In particular embodiments of this invention, the combination of the IGF-1R inhibitor and an Akt inhibitor is additive, i.e. the combination treatment regimen produces a result that is the additive effect of each constituent when it is administered alone. In accordance with this embodiment, the amount of IGF-1R inhibitor and the amount of the Akt inhibitor together constitute an effective amount to treat cancer.

In another particular embodiments of this invention, the combination of the IGF-1R inhibitor e.g., dalotuzumab and the Akt inhibitor (MK-2206) is considered therapeutically synergistic when the combination treatment regimen produces a significantly better anticancer result (e.g., cell growth arrest, apoptosis, induction of differentiation, cell death) than the additive effects of each constituent when it is administered alone at a therapeutic dose. Standard statistical analysis can be employed to determine when the results are significantly better. For example, a Mann-Whitney Test or some other generally accepted statistical analysis can be employed.

The treatment procedures of the present invention include administering the IGF 1R inhibitor (e.g., dalotuzumab) and the AKT inhibitor (MK-2206) sequentially in any order, simultaneously or a combination thereof. For example, the first treatment procedure, administration of an IGF-1R inhibitor, can take place prior to the second treatment procedure, i.e., the Akt inhibitor, after the second treatment with the Akt inhibitor, at the same time as the second treatment with the Akt inhibitor, or a combination thereof. For example, a total treatment period can be decided for the IGF-1R inhibitor. The Akt inhibitor can be administered prior to onset of treatment with the IGF-1R inhibitor or following treatment with the IGF-1R inhibitor. In addition, treatment with the Akt inhibitor can be administered during the period of IGF-1R inhibitor administration but does not need to occur over the entire IGF-1R inhibitor treatment period. In another embodiment, the treatment regimen includes pre-treatment with one agent, either the IGF-1R inhibitor or the Akt inhibitor, followed by the addition of the second agent. The administration of each component of the proposed combination therapeutic may be administered via different routes of administration.

The methods of the present invention are useful in the treatment in a wide variety of cancers, including but not limited to solid tumors (e.g., tumors of the lung, breast, colon, prostate, bladder, rectum, brain or endometrium), hematological malignancies (e.g., leukemias, lymphomas, myelomas), carcinomas (e.g. bladder carcinoma, renal carcinoma, breast carcinoma, colorectal carcinoma), neuroblastoma, or melanoma. Non-limiting examples of these cancers include cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), acute lymphocytic leukemia, acute nonlymphocytic leukemia, chronic lymphocytic leukemia, chronic myelogenous leukemia, Hodgkin's disease, non-Hodgkin's lymphoma, multiple myeloma, mesothelioma, childhood solid tumors such as brain neuroblastoma, retinoblastoma, Wilms' tumor, bone cancer and soft-tissue sarcomas, common solid tumors of adults such as head and neck cancers (e.g., oral, laryngeal and esophageal), genito urinary cancers (e.g., prostate, bladder, renal, uterine, ovarian, testicular, rectal and colon), lung cancer, breast cancer, pancreatic cancer, melanoma and other skin cancers, stomach cancer, brain cancer, liver cancer, adrenal cancer, kidney cancer, thyroid cancer, basal cell carcinoma, squamous cell carcinoma of both ulcerating and papillary type, metastatic skin carcinoma, medullary carcinoma, osteo sarcoma, Ewing's sarcoma, veticulum cell sarcoma, Kaposi's sarcoma, neuroblastoma and retinoblastoma.

In one particular embodiment of the present invention, the IGF-1R inhibitor is dalotuzumab, which can be administered in association with an AKT inhibitor (e.g., MK-2206) in further association with a further chemotherapeutic agent, such as any one or more other IGF-1R inhibitors, an alkylating agent, an antibiotic agent, an antimetabolic agent, a hormonal agent, a plant-derived agent, an anti-angiogenic agent, a differentiation inducing agent, a cell growth arrest inducing agent, an apoptosis inducing agent, a cytotoxic agent, a biologic agent, a gene therapy agent, or any combination thereof.

The combination therapy can provide a therapeutic advantage in view of the differential toxicity associated with the two treatment modalities. For example, treatment with IGF-1R inhibitors can lead to a particular toxicity that is not seen with the anti-cancer agent, and vice versa. As such, this differential toxicity can permit each treatment to be administered at a dose at which said toxicities do not exist or are minimal, such that together the combination therapy provides a therapeutic dose while avoiding the toxicities of each of the constituents of the combination agents. Furthermore, when the therapeutic effects achieved as a result of the combination treatment are enhanced or synergistic, for example, significantly better than additive therapeutic effects, the doses of each of the agents can be reduced even further, thus lowering the associated toxicities to an even greater extent.

The terms “IGF-1R”, “Insulin-like Growth Factor Receptor-I” and “Insulin-like Growth Factor Receptor, type I” are well known in the art. Although IGF-1R may be from any organism, it is preferably from an animal, more preferably from a mammal (e.g., mouse, rat, rabbit, sheep or dog) and most preferably from a human. The nucleotide and amino acid sequence of a typical human IGF-1R precursor is available at Genbank, e.g. Gene ID 3480 or NM000875. Cleavage of the precursor (e.g., between amino acids 710 and 711) produces an α-subunit and a β-subunit which associate to form a mature receptor.

“Patient” as that term is used herein, refers to the recipient of the treatment. Mammalian and non-mammalian patients are included. In a specific embodiment, the patient is a mammal, such as a human, canine, murine, feline, bovine, ovine, swine or caprine. In a particular embodiment, the patient is a human.

### Anti-IGF1R Antibodies

The present invention comprises compositions and kits comprising an anti-IGF1R antibody or antigen-binding fragment thereof in association with an AKT inhibitor (e.g., MK-2206) as well a methods of treating or preventing a hyperproliferative disorder by administering a therapeutically effective amount of the inhibitors to the patient suffering from the disorder.

In an embodiment of the invention, the anti-IGF 1R antibody or antigen-binding fragment thereof is “h7C10”, “MK-0646” or “dalotuzumab” which are used interchangeably to describe a humanized antibody that is characterized as binding IGF-1R as well as binding the IR/IGF-1 hybrid receptor. In an embodiment of the invention, the antibody or antigen-binding fragment thereof binds to the same epitope as dalotuzumab or competes for IGF1R binding with dalotuzumab.

In certain embodiments of the invention, the anti-IGF1R antibody is as described in U.S. Pat. No. 7,241,444 ('444 patent) the content of which is incorporated by reference herein in its entirety. Nucleic acid molecules for expressing the recombinant anti-IGF 1R antibodies are also described in the '444 patent.

In an embodiment of the invention, the anti-IGF1R antibody or antigen-binding fragment thereof comprises any one of more of those detailed in Table 1 below.

TABLE 1

| Inhibitor | Company |
|---|---|
| XL228 | Exelixis |
| OSI-906 | OSI |
| AMG-479 | Amgen |
| R1507 | Roche |
| Figitumumab | Pfizer |
| BMS-754807 | BMS |
| MK-0646 | Merck |
| IMC A12 | Imclone/Lilly |

Source: www.pharmastrategyblog.com/2009/06/igf1r-inhibitors-in-cancer.html

In an embodiment of the invention, the anti-IGF 1R antibody or antigen-binding fragment thereof the antibody or antigen-binding fragment thereof comprises one or more (e.g., 3) complementary determining regions derived from a non-human source and having the amino acid sequence selected from the group consisting of SEQ ID NOs: 1, 2, or 3; and a heavy chain comprising one or more (e.g., 3) complementary determining regions having an amino acid sequence selected from the group consisting of SEQ ID NOs 4, 5 or 6.

In an embodiment of the invention, the light chain immunoglobulin comprises CDR1, CDR2 and CDR3 from the amino acid sequence as set forth in SEQ ID NO: 7 or a sequence having at least 80% identity thereto after optimum alignment with the amino acid sequence set forth in SEQ ID NO: 7; and/or the heavy chain immunoglobulin comprises CDR1, CDR2 and CDR3 from the amino acid sequence as set forth in SEQ ID NO: 8 or a sequence having at least 80% identity after optimum alignment with the amino acid sequence set forth in SEQ ID NO: 8.

In an embodiment of the invention, the light chain immunoglobulin comprises the amino acid sequence as set forth in SEQ ID NO: 7 or a sequence having at least 80% identity thereto after optimum alignment with the amino acid sequence set forth in SEQ ID NO: 7; and/or the heavy chain immunoglobulin comprises the amino acid sequence as set forth in SEQ ID NO: 8 or a sequence having at least 80% identity after optimum alignment with the amino acid sequence set forth in SEQ ID NO: 8.

In an embodiment of the invention, the anti-IGF 1R comprises the light chain CDRs set forth in the light chain immunoglobulin comprising the amino acid sequence

| (SEQ ID NO: 7) | |
|---|---|
| 1 | DIVMTQSPLS LPVTPGEPAS ISCRSSQSIV HSNGNTYLQW YLQKPGQSPQ |
| 51 | LLIYKVSNRL YGVPDRFSGS GSGTDFTLKI SRVEAEDVGV YYCFQGSHVP |
| 101 | WTFGQGTKVE IKRTVAAPSV FIFPPSDEQL KSGTASVVCL LNNFYPREAK |
| 151 | VQWKVDNALQ SGNSQESVTE QDSKDSTYSL SSTLTLSKAD YEKHKVYACE |
| 201 | VTHQGLSSPV TKSFNRGEC |

and/or, the heavy chain CDRs set forth in the heavy chain immunoglobulin comprising the amino acid sequence:

| (SEQ ID NO: 8) | |
|---|---|
| 1 | QVQLQESGPG LVKPSETLSL TCTVSGYSIT GGYLWNWIRQ PPGKGLEWIG |
| 51 | YISYDGTNNY KPSLKDRVTI SRDTSKNQFS LKLSSVTAAD TAVYYCARYG |
| 101 | RVFFDYWGQG TLVTVSSAST KGPSVFPLAP SSKSTSGGTA ALGCLVYDYF |
| 151 | PEPVTVSWNS GALTSGVHTF PAVLQSSGLY SLSSVVTVPS SSLGTQTYIC |
| 201 | NVNHKPSNTK VDKRVEPKSC DKTHTCPPCP APELLGGPSV FLFPPKPKDT |
| 251 | LMISRTPEVT CVVVDVSHED PEVKFNWYVD GVEVHNAKTK PREEQYNSTY |
| 301 | RVVSVLTVLH QDWLNGKEYK CKVSNKALPA PIEKTISKAK GQPREPQVYT |
| 351 | LPPSREEMTK NQVSLTCLVK GFYPSDIAVE WESNGQPENN YKTTPPVLDS |
| 401 | DGSFFLYSKL TVDKSRWQQG NVFSCSVMHE ALHNHYTQKS LSLSPGK |

In an embodiment of the invention:

LCDR1 comprises the amino acid sequence: RSSQSIVHSNGNTYLQ (SEQ ID NO: 1);
LCDR2 comprises the amino acid sequence: KVSNRLY (SEQ ID NO: 2);
LCDR3 comprises the amino acid sequence: FQGSHVPWT (SEQ ID NO: 3); and/or;
HCDR1 comprises the amino acid sequence: GGYLWN (SEQ ID NO: 4);
HCDR2 comprises the amino acid sequence: YISYDGTNNYKPSLKD (SEQ ID NO: 5);
HCDR3 comprises the amino acid sequence: YGRVFFDY (SEQ ID NO: 6).

As stated above, the scope of the present invention comprises any antibody or antigen-binding fragment thereof comprising one or more CDRs (3 light chain CDRs and/or 3 heavy chain CDRs) and/or framework regions of any of the light chain immunoglobulin or heavy chain immunoglobulins set forth herein as identified by any of the methods set forth in Chothia et al., J. Mol. Biol. 186:651-663 (1985); Novotny and Haber, Proc. Natl. Acad. Sci. USA 82:4592-4596 (1985) or Kabat, E. A. et al., Sequences of Proteins of Immunological Interest, National Institutes of Health, Bethesda, Md., (1987)).

An “antibody” is a tetrameric molecule. In a naturally-occurring antibody, each tetramer is composed of two identical pairs of polypeptide chains, each pair having one “light” (about 25 kDa) and one “heavy” chain (about 50 70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, DELTA, gamma, alpha, or epsilon, and define the antibody's isotype as IgM, IgD, IgG (e.g., IgG1, IgG2, IgG3 or IgG4), IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact antibody has two binding sites.

Antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies (scFv), diabodies and polypeptides that contain at least a portion of an immunoglobulin that is sufficient to confer specific antigen binding to the polypeptide.

As detailed herein, an aspect of the present invention is directed to a method of improving the anti-tumor efficacy of an anti-cancer agent by co-administering an IGF-1R inhibitor and an Akt inhibitor to a patient with cancer. It is understood that the IGF-1R inhibitor for practicing the methods of the present invention are not limited to an IGF-1R specific antibody e.g., dalotuzumab. As well, even when the IGF-1R inhibitor is dalotuzumab, it is understood that it may be combined with another IGF-1R antibody or small molecule IGF-1R inhibitor. Thus, the IGF-1R inhibitor need not be limited to an antibody and instead it may comprise any IGF-1R inhibiting moiety. See, for example, Table 1. In certain embodiments, the combination therapeutic may comprise more than one IGF-1R inhibitor thus comprising an anti-IGF-1R antibody combined with a chemotherapy cocktail comprising at least two or more chemotherapeutic agents which do not significantly increase incident occurrences of adverse events, when compared with the chemotherapeutic alone.

“Nucleic acid” or a “nucleic acid molecule” as used herein refers to any DNA or RNA molecule, either single- or double-stranded and, if single-stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. In some embodiments of the invention, nucleic acids are “isolated.” This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

Nucleic acids of the invention also include fragments of the nucleic acids of the invention. A “fragment” refers to a nucleic acid sequence that is preferably at least about 10 nucleic acids in length, more preferably about 40 nucleic acids, and most preferably about 100 nucleic acids in length. A “fragment” can also mean a stretch of at least about 100 consecutive nucleotides that contains one or more deletions, insertions, or substitutions. A “fragment” can also mean the whole coding sequence of a gene and may include 5′ and 3′ untranslated regions.

The antibodies and antigen-binding fragments thereof for use in the present invention include, but are not limited to, monoclonal antibodies, synthetic antibodies, polyclonal antibodies, multispecific antibodies (including bi-specific antibodies), human antibodies, humanized antibodies, chimeric antibodies, single-chain Fvs (scfv) (including bi-specific scFvs), single chain antibodies, Fab fragments, F(abt) fragments, disulfide-linked Fvs (sdFv), and antigen-binding fragments of any of the above. In particular, antibodies for use in the present invention include immunoglobulin molecules and immunologically active portions of immunoglobulin molecules, i.e., molecules that contain an IGF-1R binding site that immunospecifically bind to IGF-1R. The immunoglobulin molecules for use in the invention can be of any type (e.g. IgG, IgE, IgM, IgD, IgA and IgY), class (e.g., IgG1, IgG2, IgG3, IgG4, IgA1 and IgA2) or subclass of immunoglobulin molecule. Preferably, the antibodies for use in the invention are IgG, more preferably, IgG1.

The antibodies for use in the invention may be from any animal origin. Preferably, the antibodies are humanized monoclonal antibodies. Alternatively, the antibodies may be fully human, e.g., so long as they bind the same epitope of the antibody claimed in the '444 patent. As used herein, “human” antibodies include antibodies having the amino acid sequence of a human immunoglobulin and include antibodies isolated from human immunoglobulin libraries or from mice or other animals that express antibodies from human genes.

Antibodies for use in the present invention may be monospecific, bispecific, trispecific or of greater multispecificity. Multispecific antibodies may immunospecifically bind to different epitopes of a polypeptide or may immunospecifically bind to both a polypeptide as well a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

The antibodies for use in the invention include derivatives of the antibodies. Standard techniques known to those of skill in the art can be used to introduce mutations in the nucleotide sequence encoding an antibody to be used with the methods for use in the invention, including, for example, site-directed mutagenesis and PCR-mediated mutagenesis which result in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, argenine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

The antibodies for use in the present invention include derivatives that are modified, i.e., by the covalent attachment of any type of molecule to the antibody. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, synthesis in the presence of tunicamycin, etc. Additionally, the derivative may contain one or more non-classical amino acids.

The present invention also provides antibodies for use in the invention that comprise a framework region known to those of skill in the art. In certain embodiments, one or more framework regions, preferably, all of the framework regions, of an antibody to be used in the compositions and methods for use in the invention are human. In certain other embodiments for use in the invention, the fragment region of an antibody for use in the invention is humanized. In certain embodiments, the antibody to be used with the methods for use in the invention is a synthetic antibody, a monoclonal antibody, an intrabody, a chimeric antibody, a human antibody, a humanized chimeric antibody, a humanized antibody, a glycosylated antibody, a multispecific antibody, a human antibody, a single-chain antibody, or a bispecific antibody.

In certain embodiments, an antibody for use in the invention has a high binding affinity for IGF-1R.

In certain embodiments, an antibody for use in the invention has a half-life in a subject, preferably a human, of about 12 hours or more, about 1 day or more, about 3 days or more, about 6 days or more, about 10 days or more, about 15 days or more, about 20 days or more, about 25 days or more, about 30 days or more, about 35 days or more, about 40 days or more, about 45 days or more, about 2 months or more, about 3 months or more, about 4 months or more, or about 5 months or more. Antibodies with increased in vivo half-lives can be generated by techniques known to those of skill in the art. For example, antibodies with increased in vivo half-lives can be generated by modifying (e.g., substituting, deleting or adding) amino acid residues identified as involved in the interaction between the Fc domain and the FcRn receptor (see, e.g., International Publication No. WO 97/34631 and U.S. patent application Ser. No. 10/020,354, entitled “Molecules with Extended Half-Lives, Compositions and Uses Thereof”, filed Dec. 12, 2001, by Johnson et al.; and U.S. Publication Nos. 2005/003700 and 2005/0064514, which are incorporated herein by reference in their entireties). Such antibodies can be tested for binding activity to antigens as well as for in vivo efficacy using methods known to those skilled in the art, for example, by immunoassays described herein.

Further, antibodies with increased in vivo half-lives can be generated by attaching to the antibodies polymer molecules such as high molecular weight polyethyleneglycol (PEG). PEG can be attached to the antibodies with or without a multifunctional linker either through site-specific conjugation of the PEG to the N- or C-terminus of the antibodies or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography. PEG-derivatized antibodies can be tested for binding activity to antigens as well as for in vivo efficacy using methods known to those skilled in the art, for example, by immunoassays described herein.

In certain embodiments, an antigen-binding fragment of an intact antibody retains capacity to bind IGF-1R. Examples include (i) a Fab fragment, a monovalent fragment consisting of the VL, VH, CL and CH1 domains; (ii) a F(ab′)2 fragment, ambivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); See, e.g., Bird et al. (1988) Science 242:423-426; and Huston et al. (1988) Proc. Natl. Acad Sci. USA 85:5879-5883). Such single chain antibodies are included by reference to the term “antibody.”

Methods of Producing Antibodies to IGF-1R are detailed, in for example, the '444 patent (supra), the content of which is incorporated by reference in its entirety.

Screening for Antibody Specificity—Techniques for generating antibodies has been described above. One may further select antibodies with certain biological characteristics, as desired. Thus, once produced, the antibodies may be screened for their binding affinity for IGF-1R. Screening for antibodies that specifically bind to IGF-1R may be accomplished using an enzyme-linked immunosorbent assay (ELISA) in which microtiter plates are coated with IGF-1R. In some embodiments, antibodies that bind IGF-1R from positively reacting clones can be further screened for reactivity in an ELISA-based assay to other IGF-1R isoforms, for example, IGF-1R using microtiter plates coated with the other IGF-1R isoform(s). Clones that produce antibodies that are reactive to another isoform of IGF-1R are eliminated, and clones that produce antibodies that are reactive to IGF-1R only may be selected for further expansion and development. Confirmation of reactivity of the antibodies to IGF-1R may be accomplished, for example, using a Western Blot assay in which protein from ovarian, breast, renal, colorectal, lung, endometrial, or brain cancer cells and purified IGF-1R and other IGF-1R isoforms are run on an SDS-PAGE gel, and subsequently are blotted onto a membrane. The membrane may then be probed with the putative anti-IGF-1R antibodies. Reactivity with IGF-1R and not another insulin-like receptor isoform confirms specificity of reactivity for IGF-1R.

General methods for detecting IGF-1R or its Derivatives—The assaying method for detecting IGF-1R using the antibodies of the invention or binding fragments thereof are not particularly limited. Any assaying method can be used, so long as the amount of antibody, antigen or antibody-antigen complex corresponding to the amount of antigen (e.g., the level of IGF-1R) in a fluid to be tested can be detected by chemical or physical means and the amount of the antigen can be calculated from a standard curve prepared from standard solutions containing known amounts of the antigen. Representative immunoassays encompassed by the present invention include, but are not limited to, those described in U.S. Pat. No. 4,367,110 (double monoclonal antibody sandwich assay); Wide et al., Kirkham and Hunter, eds. Radioimmunoassay Methods, E. and S. Livingstone, Edinburgh (1970); U.S. Pat. No. 4,452,901 (western blot); Brown et al., J. Biol. Chem. 255: 4980-4983 (1980) (immunoprecipitation of labeled ligand); and Brooks et al., Clin. Exp. Immunol. 39:477 (1980) (immunocytochemistry); immunofluorescence techniques employing a fluorescently labeled antibody, coupled with light microscopic, flow cytometric, or fluorometric detection etc. See also Immunoassays for the 80's, A. Voller et al., eds., University Park, 1981, Zola, Monoclonal Antibodies: A Manual of Techniques, pp. 147-158 (CRC Press, Inc. 1987).

A typical in vitro immunoassay for detecting IGF-1R comprises incubating a biological sample in the presence of a detectably labeled anti-IGF-1R antibody or antigen binding fragment of the present invention capable of selectively binding to IGF-1R, and detecting the labeled fragment or antibody which is bound in a sample. The antibody is bound to a label effective to permit detection of the cells or portions (e.g., IGF-1R or fragments thereof liberated from hyperplastic, dysplastic and/or cancerous cells) thereof upon binding of the antibody to the cells or portions thereof. The presence of any cells or portions thereof in the biological sample is detected by detection of the label.

The biological sample may be brought into contact with, and immobilized onto, a solid phase support or carrier, such as nitrocellulose, or other solid support or matrix, which is capable of immobilizing cells, cell particles, membranes, or soluble proteins. The support may then be washed with suitable buffers, followed by treatment with the detectably-labeled anti-IGF-1R antibody. The solid phase support may then be washed with buffer a second time to remove unbound antibody. The amount of bound label on the solid support may then be detected by conventional means. Accordingly, in another embodiment of the present invention, compositions are provided comprising the monoclonal antibodies, or binding fragments thereof, bound to a solid phase support, such as described herein.

By “solid phase support” or “carrier” is intended any support capable of binding peptide, antigen or antibody. Well-known supports or carriers, include glass, polystyrene, polypropylene, polyethylene, dextran, nylon, amylases, natural and modified celluloses, polyacrylamides, agaroses, and magnetite. The nature of the carrier can be either soluble to some extent or insoluble for the purposes of the present invention. The support material can have virtually any possible structural configuration so long as the coupled molecule is capable of binding to IGF-1R or an Anti-IGF-1R antibody. Thus, the support configuration can be spherical, as in a bead, or cylindrical, as in the inside surface of a test tube, or the external surface of a rod. Alternatively, the surface can be flat, such as a sheet, culture dish, test strip, etc. Preferred supports include polystyrene beads, Those skilled in the art will know many other suitable carriers for binding antibody, peptide or antigen, or can ascertain the same by routine experimentation.

In vitro assays in accordance with the present invention also include the use of isolated membranes from cells expressing a recombinant IGF-1R, soluble fragments comprising the ligand binding segments of IGF-1R, or fragments attached to solid phase substrates. These assays allow for the diagnostic determination of the effects of either binding segment mutations and modifications, or ligand mutations and modifications, e.g., ligand analogues.

Assays For Efficacy of Combination Immunotherapy in in vivo Models—Tumor burden can be assessed at various time points after tumor challenge using techniques well known in the art. Assays for monitoring anti-tumor response and determining the efficacy of combination immunotherapy are described below. While an improved or enhanced anti-tumor response may be most dramatically observed shortly following administration of the immunotherapy, e.g. within 5-10 days, the response may be delayed in some instances, depending on factors such as the expression level of IGF-1R, the dosage and dosing frequency of the anti-IGF-1R antibody, and the relative timing of administration of the IGF-1R inhibitor (IGF-1R antibody) relative to the timing of administration of the IGF-1Ri-SAHA. Thus, any of the well known assays may be performed on biological samples harvested at various time points following treatment or administration of the combination therapeutic in order to fully assess the anti-tumor response following immunotherapy.

Monitoring Treatment—One skilled in the art is aware of means to monitor the therapeutic outcome and/or the systemic immune response upon administering a combination treatment of the present invention. In particular, the therapeutic outcome can be assessed by monitoring attenuation of tumor growth and/or tumor regression and or the level of tumor specific markers. The attenuation of tumor growth or tumor regression in response to treatment can be monitored using one or more of several end-points known to those skilled in the art including, for instance, number of tumors, tumor mass or size, or reduction/prevention of metastasis.

### Akt and Akt Pathway Inhibitors

The present invention includes a composition or kit comprising an AKT inhibitor such as MK-2206 in association with an IGF1R inhibitor such as any of those set forth herein, e.g., dalotuzumab, for example, wherein the inhibitors are formulated together in a single composition or formulated separately into separate compositions. Methods of treating or preventing a hyperproliferative disorder such as cancer by administering a therapeutically effective amount of the inhibitors to a patient, such as a human patient, are also within the scope of the present invention. In accordance with the broad aspect of the invention, provided herein are methods of effectively treating cancers e.g., without significant adverse effects to the human patient subject to the treatment. The clinical outcomes of the treatment according to the invention are unexpected, in that the combination therapeutic comprising an anti-IGF-1R inhibitor, especially an IGF-1R antibody (e.g., dalotuzumab) and an Akt inhibitor, e.g., MK-2206(

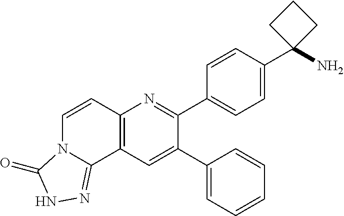

), are thought to be more effective in treating hyper-proliferative cellular disorders such as cancers that are more amenable to treatment with the combination therapeutic than treatment with each component of the combination therapeutic administered alone. As well, the combination therapeutic is more effective in treating various cancers than an Akt inhibitor by itself.

It is understood that Akt inhibitors other than MK-2206 may be combined with the IGF-1R inhibitor. Thus, an aspect of the invention includes a combination therapeutic that may comprise more than one Akt inhibitor in combination with one or more IGF-1R inhibitor. A proposed chemotherapy cocktail in accordance with this embodiment comprises at least two or more chemotherapeutic agents or anti-cancer agents (Akt inhibitors) together with at least one IGF-1R inhibitor, which do not significantly increase incident occurrences of adverse events, when compared with the chemotherapeutic alone.

Inhibitors of Akt include perifosine and are also disclosed in the following publications; WO 02/083064, WO 02/083139, WO 02/083140, US 2004-0116432, WO 02/083138, US 2004-0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344, US 2005/029941, US 2005/44294, US 2005/43361, 60/734188, 60/652,737, 60/670,469, U.S. application Ser. No. 11/999,234 and U.S. Pat. No. 7,576,209.

The anti-cancer properties attendant MK-2206 is very suitable in the proposed combination therapeutic for the treatment of cancer, in particular cancers associated with irregularities in the activity of Akt and downstream cellular targets of Akt. Such cancers include, but are not limited to, ovarian, pancreatic, breast and prostate cancer, as well as cancers (including glioblastoma) where the tumor suppressor PTEN is mutated (Cheng et al., Proc. Natl. Acad. Sci. (1992) 89:9267-9271; Cheng et al., Proc. Natl. Acad. Sci. (1996) 93:3636-3641; Bellacosa et al., Int. J. Cancer (1995) 64:280-285; Nakatani et al., J. Biol. Chem. (1999) 274:21528-21532; Graff, Expert. Opin. Ther. Targets (2002) 6(1):103-113; and Yamada and Araki, J. Cell Science. (2001) 114:2375-2382; Mischel and Cloughesy, Brain Pathol. (2003) 13(1):52-61).

Cancers that may be treated by the combination therapeutic compositions and methods of the invention include, but are not limited to: breast, prostate, colon, colorectal, lung, non-small cell lung, brain, testicular, stomach, pancrease, skin, small intestine, large intestine, throat, head and neck, oral, bone, liver, bladder, kidney, thyroid and blood. Other cancers include, advanced tumors, hairy cell leukemia, melanoma, advanced head and neck, metastatic renal cell, non-Hodgkin's lymphoma, metastatic breast, breast adenocarcinoma, advanced melanoma, pancreatic, gastric, glioblastoma, lung, ovarian, non-small cell lung, prostate, small cell lung, renal cell carcinoma, various solid tumors, multiple myeloma, metastatic prostate, malignant glioma, renal cancer, lymphoma, refractory metastatic disease, refractory multiple myeloma, cervical cancer, Kaposi's sarcoma, recurrent anaplastic glioma, and metastatic colon cancer (Dredge et al., Expert Opin. Biol. Ther. (2002) 2(8):953-966). Thus, the Akt inhibitors disclosed in the instant application are also useful in the treatment of these angiogenesis related cancers.

Further included within the scope of the invention is a method of treating or preventing a non-malignant disease in which angiogenesis is implicated. Further included within the scope of the invention is a method of treating hyperproliferative disorders which are more responsive to the combination therapeutic than each individual component acting alone.

Further included within the scope of the instant invention is the use of the instant compounds to coat stents and therefore the use of the instant combination therapeutic on coated stents for the treatment and/or prevention of restenosis (WO03/032809).

Further included within the scope of the instant invention is the use of the instant compounds for the treatment and/or prevention of osteoarthritis (WO03/035048).

The combination therapeutic compounds of the invention are also useful in preparing a medicament that is useful in treating the diseases described above, in particular cancer.

The combination therapeutic compounds of this invention may be administered to mammals, including humans, either alone or, in combination with pharmaceutically acceptable carriers, excipients or diluents, in a pharmaceutical composition, according to standard pharmaceutical practice. The combination therapeutic compounds can be administered orally or parenterally, including the intravenous, intramuscular, intraperitoneal, subcutaneous, rectal and topical routes of administration.

### Further Chemotherapeutic Agents

The instant combinations of the present invention comprising an IGF 1R inhibitor (e.g., dalotuzumab) in association with an AKT inhibitor (e.g., MK-2206) can, in an embodiment of the invention be provided in association with one or more additional therapeutic agents (e.g., anti-cancer therapeutic agents). The combinations themselves and methods of using the combinations to treat or prevent a hyperproliferative disease such as cancer are also within the scope of the present invention. Combinations of the presently disclosed combination therapeutic compounds with other therapeutic, chemotherapeutic and anti-cancer agents are also within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such agents include the following: estrogen receptor modulators, androgen receptor modulators, retinoid receptor modulators, cytotoxic/cytostatic agents, anti-proliferative agents, prenyl-protein transferase inhibitors, HMG-CoA reductase inhibitors and other angiogenesis inhibitors, HIV protease inhibitors, reverse transcriptase inhibitors, inhibitors of cell proliferation and survival signaling, bisphosphonates, aromatase inhibitors, siRNA therapeutics, γ-secretase inhibitors, agents that interfere with receptor tyrosine kinases (RTKs) and agents that interfere with cell cycle checkpoints. A combination of the present invention (e.g., dalotuzumab in association with MK-2206) may be administered to a patient with a therapeutic procedure such as surgical tumorectomy or anti-cancer radiation therapy.

“Estrogen receptor modulators” that, in an embodiment of the invention, may be provided in association with a combination of the present invention (e.g., dalotuzumab in association with MK-2206) refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” that, in an embodiment of the invention, may be provided in association with a combination of the present invention (e.g., dalotuzumab in association with MK-2206) refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” that, in an embodiment of the invention, may be provided in association with a combination of the present invention (e.g., dalotuzumab in association with MK-2206) refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” that, in an embodiment of the invention, may be provided in association with a combination of the present invention (e.g., dalotuzumab in association with MK-2206) refer to compounds which cause cell death or inhibit cell proliferation primarily by interfering directly with the cell's functioning or inhibit or interfere with cell myosis, including alkylating agents, tumor necrosis factors, intercalators, hypoxia activatable compounds, microtubule inhibitors/microtubule-stabilizing agents, inhibitors of mitotic kinesins, histone deacetylase inhibitors, inhibitors of kinases involved in mitotic progression, inhibitors of kinases involved in growth factor and cytokine signal transduction pathways, antimetabolites, biological response modifiers, hormonal/anti-hormonal therapeutic agents, haematopoietic growth factors, monoclonal antibody targeted therapeutic agents, topoisomerase inhibitors, proteosome inhibitors, ubiquitin ligase inhibitors, and aurora kinase inhibitors.

Examples of cytotoxic/cytostatic agents that, in an embodiment of the invention, may be provided in association with a combination of the present invention (e.g., dalotuzumab in association with MK-2206) include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, 4-demethoxy-3-deamino-3-aziridinyl-4-methylsulphonyl-daunorubicin (see WO 00/50032), Raf kinase inhibitors (such as Bay43-9006) and mTOR inhibitors (such as Wyeth's CCI-779).

Examples of proteosome inhibitors that, in an embodiment of the invention, may be provided in association with a combination of the present invention (e.g., dalotuzumab in association with MK-2206) include but are not limited to lactacystin and MLN-341 (Velcade).

Examples of microtubule inhibitors/microtubule-stabilising agents that, in an embodiment of the invention, may be provided in association with a combination of the present invention (e.g., dalotuzumab in association with MK-2206) include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797. In an embodiment the epothilones are not included in the microtubule inhibitors/microtubule-stabilising agents.

Some examples of topoisomerase inhibitors that, in an embodiment of the invention, may be provided in association with a combination of the present invention (e.g., dalotuzumab in association with MK-2206) are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-e]quinolin-7-one, and dimesna.

Examples of inhibitors of mitotic kinesins, and in particular the human mitotic kinesin KSP, that, in an embodiment of the invention, may be provided in association with a combination of the present invention (e.g., dalotuzumab in association with MK-2206) are described in Publications WO03/039460, WO03/050064, WO03/050122, WO03/049527, WO03/049679, WO03/049678, WO04/039774, WO03/079973, WO03/099211, WO03/105855, WO03/106417, WO04/037171, WO04/058148, WO04/058700, WO04/126699, WO05/018638, WO05/019206, WO05/019205, WO05/018547, WO05/017190, US2005/0176776. In an embodiment inhibitors of mitotic kinesins include, but are not limited to inhibitors of KSP, inhibitors of MKLP1, inhibitors of CENP-E, inhibitors of MCAK and inhibitors of Rab6-KIFL.

Examples of “histone deacetylase inhibitors” that, in an embodiment of the invention, may be provided in association with a combination of the present invention (e.g., dalotuzumab in association with MK-2206) include, but are not limited to, those disclosed in Miller, T. A. et al. J. Med. Chem. 46(24):5097-5116 (2003).

“Inhibitors of kinases involved in mitotic progression” that, in an embodiment of the invention, may be provided in association with a combination of the present invention (e.g., dalotuzumab in association with MK-2206) include, but are not limited to, inhibitors of aurora kinase, inhibitors of Polo-like kinases (PLK; in particular inhibitors of PLK-1), inhibitors of bub-1 and inhibitors of bub-R1. An example of an “aurora kinase inhibitor” is VX-680.

“Antiproliferative agents” that, in an embodiment of the invention, may be provided in association with a combination of the present invention (e.g., dalotuzumab in association with MK-2206) includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manna-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabin furanosyl cytosine, 3-aminopyridine-2-carboxaldehyde thiosemicarbazone and trastuzumab.

Examples of monoclonal antibody targeted therapeutic agents that, in an embodiment of the invention, may be provided in association with a combination of the present invention (e.g., dalotuzumab in association with MK-2206) include those therapeutic agents which have cytotoxic agents or radioisotopes attached to a cancer cell specific or target cell specific monoclonal antibody. Examples include alemtuzumab, trastuzumab, nimotuzumab, cetuximab, tositumomab, bevacizumab, or rituximab.

“HMG-CoA reductase inhibitors” that, in an embodiment of the invention, may be provided in association with a combination of the present invention (e.g., dalotuzumab in association with MK-2206) refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” that, in an embodiment of the invention, may be provided in association with a combination of the present invention (e.g., dalotuzumab in association with MK-2206) refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors that, in an embodiment of the invention, may be provided in association with a combination of the present invention (e.g., dalotuzumab in association with MK-2206) can be found in the following publications and patents; WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J of Cancer, Vol. 35, No. 9, pp. 1394-1401 (1999).

“Angiogenesis inhibitors” that, in an embodiment of the invention, may be provided in association with a combination of the present invention (e.g., dalotuzumab in association with MK-2206) refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 6, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the instant combination therapeutic compounds of the instant invention that, in an embodiment of the invention, may be provided in association with a combination of the present invention (e.g., dalotuzumab in association with MK-2206) include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in U.S. Ser. Nos. 60/310,927 (filed Aug. 8, 2001) and 60/349,925 (filed Jan. 18, 2002).

“Agents that interfere with cell cycle checkpoints” that, in an embodiment of the invention, may be provided in association with a combination of the present invention (e.g., dalotuzumab in association with MK-2206) refer to compounds that inhibit protein kinases that transduce cell cycle checkpoint signals, thereby sensitizing the cancer cell to DNA damaging agents. Such agents include inhibitors of ATR, ATM, the CHK11 and CHK12 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” that, in an embodiment of the invention, may be provided in association with a combination of the present invention (e.g., dalotuzumab in association with MK-2206) refer to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs as described by Bume-Jensen and Hunter, Nature, 411:355-365, 2001.

“Inhibitors of cell proliferation and survival signalling pathway” that, in an embodiment of the invention, may be provided in association with a combination of the present invention (e.g., dalotuzumab in association with MK-2206) refer to compounds that inhibit signal transduction cascades downstream of cell surface receptors. Such agents include inhibitors of serine/threonine kinases (including but not limited to inhibitors of Akt such as described in WO 02/083064, WO 02/083139, WO 02/083140, US 2004-0116432, WO 02/083138, US 2004-0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344, US 2005/029941, US 2005/44294, US 2005/43361, 60/734,188, 60/652,737, 60/670,469), inhibitors of Raf kinase (for example BAY-43-9006), inhibitors of MEK (for example CI-1040 and PD-098059), inhibitors of mTOR (for example Wyeth CCI-779), and inhibitors of PI3K (for example LY294002).

NSAIDs (non-steroidal anti-inflammatory drugs) that, in an embodiment of the invention, may be provided in association with a combination of the present invention (e.g., dalotuzumab in association with MK-2206) include COX-2 inhibiting agents. For purposes of this specification an NSAID is potent if it possesses an IC₅₀ for the inhibition of COX-2 of 1 μM or less as measured by cell or microsomal assays. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, U.S. Pat. No. 5,861,419, U.S. Pat. No. 6,001,843, U.S. Pat. No. 6,020,343, U.S. Pat. No. 5,409,944, U.S. Pat. No. 5,436,265, U.S. Pat. No. 5,536,752, U.S. Pat. No. 5,550,142, U.S. Pat. No. 5,604,260, U.S. Pat. No. 5,698,584, U.S. Pat. No. 5,710,140, WO 94/15932, U.S. Pat. No. 5,344,991, U.S. Pat. No. 5,134,142, U.S. Pat. No. 5,380,738, U.S. Pat. No. 5,393,790, U.S. Pat. No. 5,466,823, U.S. Pat. No. 5,633,272 and U.S. Pat. No. 5,932,598, all of which are hereby incorporated by reference.

Other examples of angiogenesis inhibitors that, in an embodiment of the invention, may be provided in association with a combination of the present invention (e.g., dalotuzumab in association with MK-2206) include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RP14610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used herein, “integrin blockers” that, in an embodiment of the invention, may be provided in association with a combination of the present invention (e.g., dalotuzumab in association with MK-2206) refers to compounds which selectively antagonize, inhibit or counteract binding of a physiological ligand to the αᵥβ₃ integrin, to compounds which selectively antagonize, inhibit or counteract binding of a physiological ligand to the αvβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, α₁β₁, α₂β₁, α₅β₁, α₆β₁ and α₆β₄ integrins.

Some specific examples of tyrosine kinase inhibitors that, in an embodiment of the invention, may be provided in association with a combination of the present invention (e.g., dalotuzumab in association with MK-2206) include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382,2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, PPAR-γ (i.e., PPAR-gamma) agonists and PPAR-δ (i.e., PPAR-delta) agonists that, in an embodiment of the invention, may be provided in association with a combination of the present invention (e.g., dalotuzumab in association with MK-2206) are useful in the treatment of certain malignancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999; 274:9116-9121; Invest. Ophthalmol Vis. Sci. 2000; 41:2309-2317). PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy) phenoxy)propoxy)-2-ethylchromane-2-carboxylic acid (disclosed in U.S. Ser. No. 60/235,708 and 60/244,697).

Another embodiment of the instant invention is the use of the presently disclosed combination therapy comprising the combination therapeutic compounds in concert with gene therapy for the treatment of cancer. For an overview of genetic strategies to treating cancer see Hall et al (Am. J. Hum. Genet. 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J. Immunol. 2000; 164:217-222).

Inherent multidrug resistance (MDR) inhibitors that, in an embodiment of the invention, may be provided in association with a combination of the present invention (e.g., dalotuzumab in association with MK-2206) include any MDR inhibitor, in particular, MDRs associated with high levels of expression of transporter proteins. Such MDR inhibitors include inhibitors of p-glycoprotein (P-gp), such as LY335979, XR9576, OC144-093, R101922, VX853 and PSC833 (valspodar).

Neurokinin-1 receptor antagonists that, in an embodiment of the invention, may be provided in association with a combination of the present invention (e.g., dalotuzumab in association with MK-2206) include those disclosed, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are incorporated herein by reference.

The combination therapeutic compounds of the instant invention (e.g., dalotuzumab in association with MK-2206) may also be provided in association with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

The combination therapeutic compounds of the instant invention (e.g., dalotuzumab in association with MK-2206) may also be provided in association with an immunologic-enhancing drug, such as levamisole, isoprinosine and Zadaxin.

The combination therapeutic compounds of the instant invention (e.g., dalotuzumab in association with MK-2206) may also be provided in association with P450 inhibitors including: xenobiotics, quinidine, tyramine, ketoconazole, testosterone, quinine, methyrapone, caffeine, phenelzine, doxorubicin, troleandomycin, cyclobenzaprine, erythromycin, cocaine, furafyline, cimetidine, dextromethorphan, ritonavir, indinavir, amprenavir, diltiazem, terfenadine, verapamil, cortisol, itraconazole, mibefradil, nefazodone and nelfinavir.

The combination therapeutic compounds of the instant invention (e.g., dalotuzumab in association with MK-2206) may also be provided in association with Pgp and/or BCRP inhibitors including: cyclosporin A, PSC833, GF120918, cremophorEL, fumitremorgin C, Ko132, Ko134, Iressa, Imatnib mesylate, EKI-785, C11033, novobiocin, diethylstilbestrol, tamoxifen, resperpine, VX-710, tryprostatin A, flavonoids, ritonavir, saquinavir, nelfinavir, omeprazole, quinidine, verapamil, terfenadine, ketoconazole, nifidepine, FK506, amiodarone, XR9576, indinavir, amprenavir, cortisol, testosterone, LY335979, OC144-093, erythromycin, vincristine, digoxin and talinolol.

The combination therapeutic compounds of the instant invention (e.g., dalotuzumab in association with MK-2206) may also be provided in association with agents that are useful for treating or preventing cancers including bone cancer, for example bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

The combination therapeutic compounds of the instant invention (e.g., dalotuzumab in association with MK-2206) may also be provided in association with agents useful for treating or preventing breast cancer such as aromatase inhibitors. Examples of aromatase inhibitors include but are not limited to: anastrozole, letrozole and exemestane.

The combination therapeutic compounds of the instant invention (e.g., dalotuzumab in association with MK-2206) may also be provided in association with siRNA or RNAi therapeutic agents.

The combination therapeutic compounds of the instant invention may also be useful for treating or preventing cancer in combination with PARP inhibitors.

The combination therapeutic compounds of the instant invention may also be useful for treating cancer in combination with the following therapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexylen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatine); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (masterone Injection®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); zoledronate (Zometa®) and vorinostat (Zolinza®).

Other agents that may be provided in association with a combination of the present invention include insulin, insulin secretagogues, PPAR-gamma agonists, metformin, somatostatin receptor agonists such as octreotide, DPP4 inhibitors, sulfonylureas, alpha-glucosidase inhibitors, potassium salts, magnesium salts, beta-blockers (such as atenolol) and endothelin-a (ETa)antagonists.

Thus, an aspect of the invention encompasses the use of the instantly claimed combination therapy methods employing combination therapeutic compounds in combination with yet another compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase inhibitors, agents that interfere with receptor tyrosine kinases (RTKs), an agent that interferes with a cell cycle checkpoint and any of the therapeutic agents listed above.

### Modes and Doses of Administration

The methods of the present invention comprise administering to a patient in need thereof a first amount of an IGF-1R inhibitor, e.g., preferably an IGF-1R antibody exemplified by dalotuzumab, in a first treatment procedure, and a second amount of an Akt inhibitor in a second treatment procedure. The first and second treatments together comprise a therapeutically effective amount. It is understood that administration of IGF-1R inhibitor and the Akt inhibitor are interchangeable in that the first treatment protocol may comprise an Akt inhibitor followed by the second treatment protocol comprising an IGF-1R inhibitor.

When a composition according to this invention is administered into a human subject, the daily dosage will normally be determined by the prescribing physician with the dosage generally varying according to the age, weight, and response of the individual patient, as well as the severity of the patient's symptoms.

The dosage regimen utilizing the combination therapeutic combination compounds of the instant invention can be selected in accordance with a variety of factors including type, species, age, weight, sex and the type of cancer being treated; the severity (i.e., stage) of the cancer to be treated; the route of administration; the renal and hepatic function of the patient; and the particular compound or salt thereof employed. An ordinarily skilled physician or veterinarian can readily determine and prescribe the effective amount of the drug required to treat, for example, to prevent, inhibit (fully or partially) or arrest the progress of the disease.

For example, the combination therapeutic compounds of the instant invention can be administered once daily (QD), or divided into multiple daily doses such as twice daily (BID), and three times daily (TID).

In addition, the administration can be continuous, i.e., every day, or intermittently. The terms “intermittent” or “intermittently” as used herein means stopping and starting at either regular or irregular intervals. For example, intermittent administration of a compound of the instant invention may be administration one to six days per week or it may mean administration in cycles (e.g. daily administration for two to eight consecutive weeks, then a rest period with no administration for up to one week) or it may mean administration on alternate days.

In addition, the combination therapeutic compounds of the instant invention may be administered according to any of the schedules described herein, consecutively for a few weeks, followed by a rest period. For example, the combination therapeutic compounds of the instant invention may be administered according to any one of the schedules described above from two to eight weeks, followed by a rest period of one week, or twice daily at a dose of 100-500 mg for three to five days a week. In another particular embodiment, the AKT inhibitor component of the combination therapeutic compounds of the instant invention may be administered three times daily for two consecutive weeks, followed by one week of rest.

Any one or more of the specific dosages and dosage schedules of the compounds of the instant invention, may also be applicable to any one or more of the therapeutic agents to be used in the combination treatment (hereinafter referred to as the “second therapeutic agent”). Moreover, the specific dosage and dosage schedule of this subsequent therapeutic agent can further vary, and the optimal dose, dosing schedule and route of administration will be determined based upon the specific second therapeutic agent that is being used.

Of course, the route of administration of the compounds of the instant invention is independent of the route of administration of the second therapeutic agent. In an embodiment, the administration for a compound of the instant invention is oral administration. In another embodiment, the administration for a compound of the instant invention is intravenous administration. Thus, in accordance with these embodiments, a compound of the instant invention is administered orally or intravenously, and the second therapeutic agent can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

In addition, a compound of the instant invention and second therapeutic agent may be administered by the same mode of administration, i.e. both agents administered e.g. orally, by IV. However, it is also within the scope of the present invention to administer a compound of the instant invention by one mode of administration, e.g. oral, and to administer the second therapeutic agent by another mode of administration, e.g. IV or any other ones of the administration modes described hereinabove.

The first treatment procedure, administration of a compound of the instant invention, can take place prior to the second treatment procedure, i.e., the second therapeutic agent, after the treatment with the second therapeutic agent, at the same time as the treatment with the second therapeutic agent, or a combination thereof. For example, a total treatment period can be decided for a compound of the instant invention. The second therapeutic agent can be administered prior to onset of treatment with a compound of the instant invention or following treatment with a compound of the instant invention. In addition, anti-cancer treatment e.g., Akt inhibitor can be administered during the period of administration of a compound of the instant invention but does not need to occur over the entire treatment period of a compound of the instant invention.

### Administration of the IGF-1R Inhibitor

### Dose and Route of Administration

The combination therapeutic comprising IGF-1R inhibitors (IGF1Ris), e.g., antibodies and antigen-binding fragments thereof (e.g., dalotuzumab) and chemotherapeutic agents of the invention are administered to a human patient, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Intravenous or subcutaneous administration of the antibody is preferred. Three distinct delivery approaches are expected to be useful for delivery of the antibodies in accordance with the invention. Conventional intravenous delivery will presumably be the standard delivery technique for the majority of turnouts. However, in connection with some tumours, such as those in the peritoneal cavity exemplified by tumours of the ovaries, biliary duct, other ducts, and the like, intraperitoneal administration may prove favorable for obtaining high dose of antibody at the tumour and to minimize antibody clearance. In a similar manner certain solid tumours possess vasculature that is appropriate for regional perfusion. Regional perfusion will allow the obtention of a high dose of the antibody at the site of a tumour and will minimize short term clearance of the antibody. Likewise, the IGF-1R is can be administered in intravenous (bolus or infusion), intraperitoneal, subcutaneous, or intramuscular form, all using forms well known to those of ordinary skill in the pharmaceutical arts.

The IGF-1R is can also be administered in the form of a depot injection or implant preparation, which may be formulated in such a manner as to permit a sustained release of the active ingredient. The active ingredient can be compressed into pellets or small cylinders and implanted subcutaneously or intramuscularly as depot injections or implants. Implants may employ inert materials such as biodegradable polymers or synthetic silicones, for example, Silastic, silicone rubber or other polymers manufactured by the Dow-Corning Corporation.

The IGF-1Ri can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

As with any protein or antibody infusion based therapeutic, safety concerns are related primarily to (i) cytokine release syndrome, i.e., hypotension, fever, shaking, chills, (ii) the development of an immunogenic response to the material (i.e., development of human antibodies by the patient to the antibody therapeutic, or HAHA or HACA response), and (iii) toxicity to normal cells that express the EGF receptor, e,g., hepatocytes which express EGFR and/or IGF-1R. Standard tests and follow up will be utilized to monitor each of these safety concerns. In particular, liver function will be monitored frequently during clinical trails in order to assess damage to the liver, if any.

For the prevention or treatment of disease, the appropriate dosage of antibody will depend on the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. In a combination therapy regimen, the combination therapeutic disclosed herein is administered in a therapeutically effective or synergistic amount. As used herein, a “therapeutically effective” amount is such that co-administration of anti-IGF-1R antibody and one or more other therapeutic agents, or administration of a composition of the present invention, results in reduction or inhibition of the targeting disease or condition. A “therapeutically synergistic” amount is that amount of anti-IGF-1R antibody and one or more other therapeutic agents necessary to synergistically or significantly reduce or eliminate conditions or symptoms associated with a particular disease.

Intravenously or subcutaneously, the patient would receive the IGF-1R inhibitor in quantities sufficient to deliver between about 3-1500 mg/m² per day, for example, about 3, 30, 60, 90, 180, 300, 600, 900, 1200 or 1500 mg/m² per day. Such quantities may be administered in a number of suitable ways, e.g. large volumes of low concentrations of IGF-1Ri during one extended period of time or several times a day. The quantities can be administered for one or more consecutive days, intermittent days or a combination thereof per week (7 day period). Alternatively, low volumes of high concentrations of IGF-1Ri during a short period of time, e.g. once a day for one or more days either consecutively, intermittently or a combination thereof per week (7 day period). For example, a dose of 300 mg/m² per day can be administered for 5 consecutive days for a total of 1500 mg/m² per treatment. In another dosing regimen, the number of consecutive days can also be 5, with treatment lasting for 2 or 3 consecutive weeks for a total of 3000 mg/m² and 4500 mg/m² total treatment.

Typically, an intravenous formulation may be prepared which contains a concentration of the IGF-1R inhibitor of between about 1.0 mg/mL to about 10 mg/mL, e.g. 2.0 mg/mL, 3.0 mg/mL, 4.0 mg/mL, 5.0 mg/mL, 6.0 mg/mL, 7.0 mg/mL, 8.0 mg/mL, 9.0 mg/mL and 10 mg/mL and administered in amounts to achieve the doses described above. In one example, a sufficient volume of intravenous formulation can be administered to a patient in a day such that the total dose for the day is between about 300 and about 1500 mg/m².

Subcutaneous formulations, preferably prepared according to procedures well known in the art at a pH in the range between about 5 and about 12, also include suitable buffers and isotonicity agents, as described below. They can be formulated to deliver a daily dose of IGF-1Ri in one or more daily subcutaneous administrations, e.g., one, two or three times each day.

A broad aspect of the invention involves the combined administration of an anti-IGF-1R antibody and one or more chemotherapeutic agents, preferably an Akt inhibitor, more preferably MK-2206. The combined administration includes co administration, using separate formulations or a single pharmaceutical formulation, and consecutive administration in either order, wherein preferably there is a time period while both (or all) active agents simultaneously exert their biological activities. Preparation and dosing schedules for such chemotherapeutic agents may be used according to manufacturers' instructions or as determined empirically by the skilled practitioner. Preparation and dosing schedules for chemotherapy are also described in Chemotherapy Service Ed., M. C. Perry, Williams & Wilkins, Baltimore, Md. (1992). The chemotherapeutic agent or anti-cancer agent may precede, or follow administration of the antibody or may be given simultaneously therewith. The clinical dosing of therapeutic combination of the present invention are likely to be limited by the extent of adverse reactions skin rash as observed with monoclonal anti-IGF-1R antibodies and an PI3/Akt pathway inhibitor used in the clinic today.

Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved. Further information about suitable dosages is provided in the Example below.

Depending on the type and severity of the disease, about 1 .μ.g/kg to 50 mg/kg (e.g. 0.1-20 mg/kg) of antibody is an initial candidate dosage for administration to the patient, whether, for example, by one or more separate administrations, or by continuous infusion. A typical daily dosage might range from about 1 .mu.g/kg to about 100 mg/kg or more, depending on the factors mentioned above. For repeated administrations over several days or longer, depending on the condition, the treatment is sustained until a desired suppression of disease symptoms occurs. However, other dosage regimens may be useful.

In one aspect, the antibody of the invention is administered bi-weekly, weekly or may be administered every two to three weeks, at a dose ranged from about 5 mg/kg to about 15 mg/kg. More preferably, such dosing regimen is used in combination with a chemotherapy regimen for treating erlotinib resistant cancers such as NSCLC. In some aspects, the chemotherapy regimen involves the traditional high-dose intermittent administration. In some other aspects, the chemotherapeutic agents are administered using smaller and more frequent doses without scheduled breaks (“metronomic chemotherapy”). The progress of the therapy of the invention is easily monitored by conventional techniques and assays.

In one embodiment, the dosing sequence comprises administering dalotuzumab concurrently with the Akt inhibitor—IGF-1R is administered once a week at a dose of from 5 mg/kg, preferably 10-30 mg/kg and most preferably, 20 mg/kg once weekly together with the Akt inhibitor, wherein the IGF-1R inhibitor is administered via injection and the Akt inhibitor is administered orally. Alternatively, both agents may be administered via injection. In alternative embodiments, MK-2206 may be administered orally while the anti-IGF1R antibody (e.g., dalotuzumab) is administered at the same time via injection. In other alternatives, the IGF-1R antibody is administered at a dose of 20 mg/kg i.v weekly while the Akt inhibitor may be administered at 60 mg once every other day. In another alternative, the IGF-1R antibody is administered at a dose of 10 or 20 mg/kg i.v weekly while the Akt inhibitor may be administered at 200 mg once weekly.

Alternative dosing regiment for dalotuzumab is as follows:

(i) 15 mg/kg loading, followed by 7.5 mg/kg every week.

(ii) 20 mg/kg every other week

(iii) 20 mg/kg once a week

(iii) 10 mg/kg weekly or every other week

(iv) 30 mg/kg every three weeks

For parenteral administration, the antibody can be formulated as a solution, suspension, emulsion or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by known or suitable techniques. The administration of the combination therapeutic may continue until disease progression.

Administration of the Akt inhibitor

### Dose and Route of Administration

The Akt inhibitor (e.g., MK-2206) for use in the methods of the invention can be administered once daily (QD), or divided into multiple daily doses such as twice daily (BID), and three times daily (TID). More, the Akt inhibiting compounds of the instant invention can be administered at a total daily dosage of up to 10,000 mg, e.g., 2,000 mg, 3,000 mg, 4,000 mg, 6,000 mg, 8,000 mg or 10,000 mg, which can be administered in one daily dose or can be divided into multiple daily doses as described above.

In addition, the administration can be continuous, i.e., every day, or intermittently. The terms “intermittent” or “intermittently” as used herein means stopping and starting at either regular or irregular intervals. For example, intermittent administration of a compound of the instant invention may be administration one to six days per week or it may mean administration in cycles (e.g. daily administration for two to eight consecutive weeks, then a rest period with no administration for up to one week) or it may mean administration on alternate days.

In addition, the Akt inhibiting compounds for use in the methods of instant invention may be administered according to any of the schedules described herein, consecutively for a few weeks, followed by a rest period. For example, the Akt inhibitor may be administered according to any one of the schedules described above from two to eight weeks, followed by a rest period of one week, or twice daily at as a therapeutic dose of 100-500 mg for three to five days a week. In another particular embodiment, the compounds of the instant invention may be administered three times daily for two consecutive weeks, followed by one week of rest.

A currently preferred treatment protocol comprises administration of the Akt inhibitor either once every other day at a dose of 60 mg together with or concurrently with the IGF-1R inhibitor. Alternatively, it may be administered once a week at a dosed of 200 mg.

It should be apparent to a person skilled in the art that the various modes of administration, dosages and dosing schedules described herein merely set forth specific embodiments and should not be construed as limiting the broad scope of the invention. Any permutations, variations and combinations of the dosages and dosing schedules are included within the scope of the present invention.

### Combination Administration

The first treatment procedure, administration of an IGF-1R inhibitor (e.g., dalotuzumab), can take place prior to the second treatment procedure, after the treatment with the Akt inhibitor (e.g, MK-2206), at the same time as the treatment with the Akt inhibitor or a combination thereof. It is also within the scope of the invention to administer a component of the combination therapeutic by one mode of administration, e.g., IV injection especially where one component of the combination therapeutic is a large molecule e.g., antibody and to administer the second or subsequent therapeutic component or agent by another mode of administration e.g., oral.

Preferably, the method according to the invention comprises administering to a patient in need thereof a combination therapeutic comprising an IGF-1R inhibitor and an Akt inhibitor, wherein the Akt inhibitor e.g., MK-2206 is preferably administered once every other day (QOD) at a dose of 60 mg. Alternatively or in addition thereto, the Akt inhibitor may be administered once a week (QWK) at dose of 200 mg. IGF-1R inhibitor or any one of the Akt inhibitors s can be administered in accordance with any dose and dosing schedule that, together with the effect of the anti-cancer agent, achieves a dose effective to treat cancer.

### Pharmaceutical Compositions

The invention also encompasses pharmaceutical compositions comprising pharmaceutically acceptable salts of the IGF-1R is (e.g., dalotuzumab) and/or the anti-cancer agents. The preparation of pharmaceutical compositions that contain an active component is well understood in the art, for example, by mixing, granulating, or tablet-forming processes. The active therapeutic ingredient is often mixed with excipients that are pharmaceutically acceptable and compatible with the active ingredient. For oral administration, the active agents are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions and the like as detailed above.

The amount of the combination therapeutic or any one or more of its component compounds administered to the patient is less than an amount that would cause toxicity in the patient. In the certain embodiments, the amount of each compound that is administered to the patient is less than the amount that causes a concentration of the compound in the patient's plasma to equal or exceed the toxic level of the compound. The optimal amount of the combination therapeutic or each component of the combination therapeutic compound that should be administered to the patient in the practice of the present invention will depend on the particular compound used and the type of cancer being treated.

Suitable pharmaceutically acceptable salts of the compounds described herein and suitable for use in the method of the invention, are conventional non-toxic salts and can include a salt with a base or an acid addition salt such as a salt with an inorganic base, for example, an alkali metal salt (e.g., lithium salt, sodium salt, potassium salt, etc.), an alkaline earth metal salt (e.g., calcium salt, magnesium salt, etc.), an ammonium salt; a salt with an organic base, for example, an organic amine salt (e.g., triethylamine salt, pyridine salt, picoline salt, ethanolamine salt, triethanolamine salt, dicyclohexylamine salt, N,N′-dibenzylethylenediamine salt, etc.) etc.; an inorganic acid addition salt (e.g., hydrochloride, hydrobromide, sulfate, phosphate, etc.); an organic carboxylic or sulfonic acid addition salt (e.g., formate, acetate, trifluoroacetate, maleate, tartrate, methanesulfonate, benzenesulfonate, p-toluenesulfonate, etc.); a salt with a basic or acidic amino acid (e.g., arginine, aspartic acid, glutamic acid, etc.) and the like.

The invention also encompasses pharmaceutical compositions comprising hydrates of the IGF-1R is and/or the anti-cancer agents. The term “hydrate” includes but is not limited to hemihydrate, monohydrate, dihydrate, trihydrate and the like.

In addition, this invention also encompasses pharmaceutical compositions comprising any solid or liquid physical form of dalotuzumab or any of the other IGF-1R inhibitors. For example, the IGF-1R is can be in a crystalline form, in amorphous form, and have any particle size. The IGF-1Ri particles may be micronized, or may be agglomerated, particulate granules, powders, oils, oily suspensions or any other form of solid or liquid physical form.

For oral administration, the pharmaceutical compositions can be liquid or solid. Suitable solid oral formulations include tablets, capsules, pills, granules, pellets and the like. Suitable liquid oral formulations include solutions, suspensions, dispersions, emulsions, oils and the like.

Any inert excipient that is commonly used as a carrier or diluent may be used in the formulations of the present invention, such as for example, a gum, a starch, a sugar, a cellulosic material, an acrylate, or mixtures thereof. The compositions may further comprise a disintegrating agent and a lubricant, and in addition may comprise one or more additives selected from a binder, a buffer, a protease inhibitor, a surfactant, a solubilizing agent, a plasticizer, an emulsifier, a stabilizing agent, a viscosity increasing agent, a sweetener, a film forming agent, or any combination thereof. Furthermore, the compositions of the present invention may be in the form of controlled release or immediate release formulations.

The IGF-1R inhibitor can be administered as active ingredients in admixture with suitable pharmaceutical diluents, excipients or carriers (collectively referred to herein as “carrier” materials or “pharmaceutically acceptable carriers”) suitably selected with respect to the intended form of administration. As used herein, “pharmaceutically acceptable carrier” is intended to include any and all solvents, dispersion media, coatings, antibacterial and antifungal agents, isotonic and absorption delaying agents, and the like, compatible with pharmaceutical administration.

Suitable carriers are described in the most recent edition of Remington's Pharmaceutical Sciences, a standard reference text in the field, which is incorporated herein by reference.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil. Solutions or suspensions can also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide.

Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), a glidant (e.g., colloidal silicon dioxide), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film foaming agents (e.g., ethyl cellulose, acrylates, polymethacrylates) and/or adjuvants.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral compositions in dosage unit form for ease of administration and uniformity of dosage, Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions comprising the therapeutic combination or any one or more of the individual components of the combination of the invention may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane dial. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables. The pharmaceutical compositions comprising the therapeutic combination or any one or more of the individual components of the combination of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

For IV administration, Glucuronic acid, L-lactic acid, acetic acid, citric acid or any pharmaceutically acceptable acid/conjugate base with reasonable buffering capacity in the pH range acceptable for intravenous administration can be used as buffers. Sodium chloride solution wherein the pH has been adjusted to the desired range with either acid or base, for example, hydrochloric acid or sodium hydroxide, can also be employed. Typically, a pH range for the intravenous formulation can be in the range of from about 5 to about 12. A referred pH range for intravenous formulation comprising an IGF-1Ri, wherein the IGF-1Ri has a hydroxamic acid moiety, can be about 9 to about 12.

Subcutaneous formulations, preferably prepared according to procedures well known in the art at a pH in the range between about 5 and about 12, also include suitable buffers and isotonicity agents. They can be formulated to deliver a daily dose of the active agent in one or more daily subcutaneous administrations. The choice of appropriate buffer and pH of a formulation, depending on solubility of the IGF-1Ri to be administered, is readily made by a person having ordinary skill in the art. Sodium chloride solution wherein the pH has been adjusted to the desired range with either acid or base, for example, hydrochloric acid or sodium hydroxide, can also be employed in the subcutaneous formulation. Typically, a pH range for the subcutaneous formulation can be in the range of from about 5 to about 12. A preferred pH range for subcutaneous formulation of an IGF-1Ri a hydroxamic acid moiety, can be about 9 to about 12.

The pharmaceutical compositions comprising the therapeutic combination (dalotuzumab+MK-2206) or any one or more of the individual components of the combination of the invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

Any one or more components of the combination therapeutic compounds for use in the proposed combination treatment of the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Combination therapeutic compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

Included in the instant invention is the free form of the Akt inhibitors, especially small molecule inhibitors of the Akt pathway including MK-2206, as well as the pharmaceutically acceptable salts and stereoisomers thereof. The term “free form” refers to the amine compounds in non-salt form. The encompassed pharmaceutically acceptable salts not only include the isolated salts exemplified for the specific compound described herein (MK-2206), but also all the typical pharmaceutically acceptable salts of the free form of this (MK-2206) and other small molecule inhibitors of the Akt pathway. The free form of the specific salt compound of MK 2206 for example, may be isolated using techniques known in the art. See for example, Ser. No. 11/999,234 and U.S. Pat. No. 7,576,209 (the '209 patent), which describes, inter alia, compounds of Formula A and methods of generating free forms thereof. The contents

The pharmaceutically acceptable salts of the instant compound useful in practicing the methods of the instant invention as well as that of compound A are also disclosed in the '209 patent. Thus, pharmaceutically acceptable salts of MK-2206 include the conventional non-toxic salts of the compound as formed by reacting a basic instant compound with an inorganic or organic acid. See the 209 patent. When the specific compound useful in practicing the method of the invention e.g., MK-22-6 is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The preparation of the pharmaceutically acceptable salts described above and other typical pharmaceutically acceptable salts is more fully described by Berg et al., “Pharmaceutical Salts,” J. Pharm. Sci., 1977:66:1-19.

Potentially internal salts or zwitterions of the Akt inhibitors e.g., MK-2206 are also encompassed by the invention, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

The IGF-1R inhibitor and the Akt inhibitor can be formulated in the same formulation for simultaneous administration, or they can be in two separate dosage forms, which may be administered simultaneously or sequentially as described above.

The pharmaceutical compositions can be included in a container, pack, or dispenser together with instructions for administration.

Although the methods of the present invention can be practiced in vitro, it is contemplated that the preferred embodiment for the methods of selectively inducing cell death, terminal differentiation, cell growth arrest and/or apoptosis of neoplastic cells will comprise contacting the cells in vivo, i.e., by administering the compounds to a subject harboring neoplastic cells or tumor cells in need of treatment.

The invention is illustrated in the examples in the Experimental Detail Section that follows. This section is set forth to aid in an understanding of the invention but is not intended to, and should not be construed to limit in any way the invention as set forth in the claims which follow thereafter.

### EXAMPLES

RTK/PI3K/AKT signaling promotes cell proliferation and cell survival in multiple cancer-types. Inhibition of this pathway is an effective cancer therapy. Signaling pathways such as PI3K/AKT have been demonstrated to have feedback loops that regulate signaling intensity. The present invention demonstrates the existence of a novel negative feedback loop that responds to AKT-inhibition with MK-2206. In multiple cancer cell lines in vitro and in vivo, multiple RTKs were activated in response to AKT inhibition. The AKT-mediated negative feedback appears to be functionally important, Blockade of this feedback loop with an IGF-1R inhibitor, e.g., dalotuzumab, substantially increased the efficacy of the AKT inhibitor, both in vitro and in vivo. Therefore, the AKT inhibitor, MK-2206, in combination with RTK-inhibitors may provide benefit to cancers that utilize the feedback loop as a source of resistance to AKT inhibition.

### Rationale

AKT inhibition has been shown both in vitro and in vivo to play an important role in cell proliferation and/or survival. The allosteric AKT inhibitor, MK-2206, potently inhibits AKT kinase activity. Treatment of a broad spectrum of cancer cell lines in vitro and in vivo has demonstrated that monotherapy is efficacious. In an effort to further increase MK-2206 efficacy, it was combined with the IGF-1R antibody dalotuzumab. In this study, the inventors report on the surprising discovery that MK-2206 treatment results in upregulation of multiple RTKs, including IGF-1R. This upregulation appears to play a functional role in determining efficacy since combining the IGF-1R inhibitor, e.g., dalotuzumab with an Akt inhibitor e.g., MK-2206 increases efficacy in vitro and in vivo.

### Akt Inhibition Results in the Upregulation of Phosphorylation of Multiple RTKs

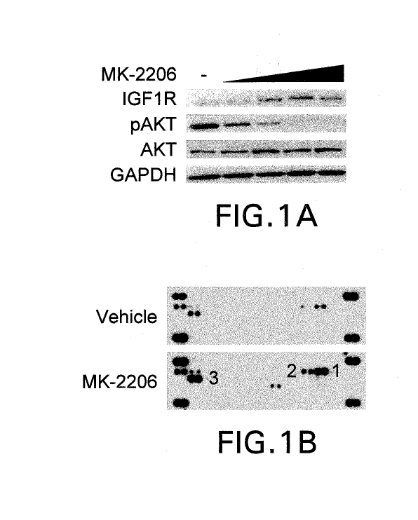

In the course of studying the signaling effects mediated by AKT inhibition with MK-2206, it was observed that the IGF-1R tyrosine kinase was upregulated. To study this phenomenon in more detail, the AKT inhibitor, MK-2206 was titrated on the colorectal cancer cell line HT-29. As shown in FIG. 1A, phosphorylation of AKT is diminished in a dose dependent manner. Surprisingly, IGF-1R levels increase in a dose dependent manner. To determine if the upregulation in total levels of IGF-1R also corresponded to an increase in activity as determined by receptor phosphorylation, phospho-RTK arrays were used. As shown in FIG. 1B, MK-2206 treatment caused an increase in phosphorylation of IGF-1R. Interestingly, phosphorylation of IR and cMET was also observed. Together these results demonstrate that AKT inhibition with MK-2206 causes and increase in the total levels of IGF-1R as well as an increase in phosphorylation of IGF-1R, IR, and cMET.

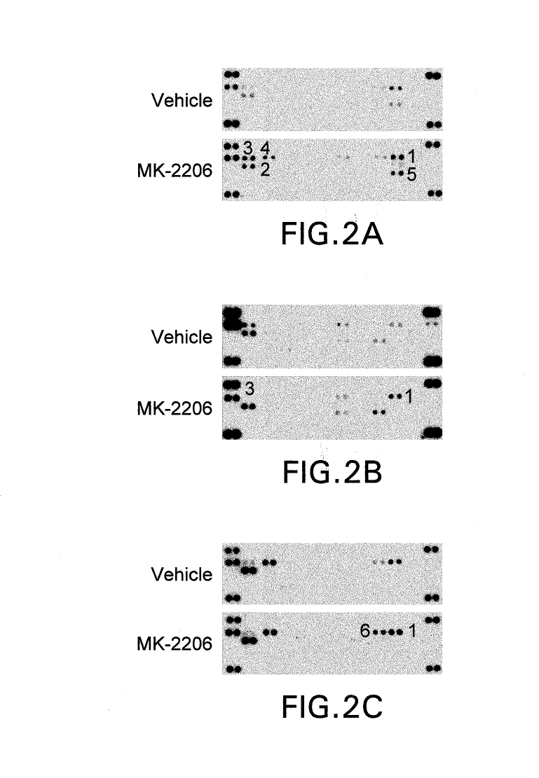

To investigate whether the above upregulation of RTKs occurs in additional cell lines/cancer types, cell lines from different cancer types (colon, breast, lung) were treated with MK-2206 and then phosphorylation of RTKs were assessed using phospho-RTK arrays. As shown in FIG. 2, MK-2206 treatment results in increasing the phosphorylation of multiple RTKs in multiple cancer cell lines. The RTKs, IGF-1R, IR, cMET, Her2, Her3, and EphA2 are shown to increase in phosphorylation in a cell type-dependent manner. These experiments also revealed that RTK activity (i.e. Her2) can decrease. Together, these results demonstrate that the activity of multiple RTKs is affected by AKT inhibition in a cell type-dependent manner.

Co-Inhibition of Akt and IGF-1R is More Efficacious than Individual Treatments

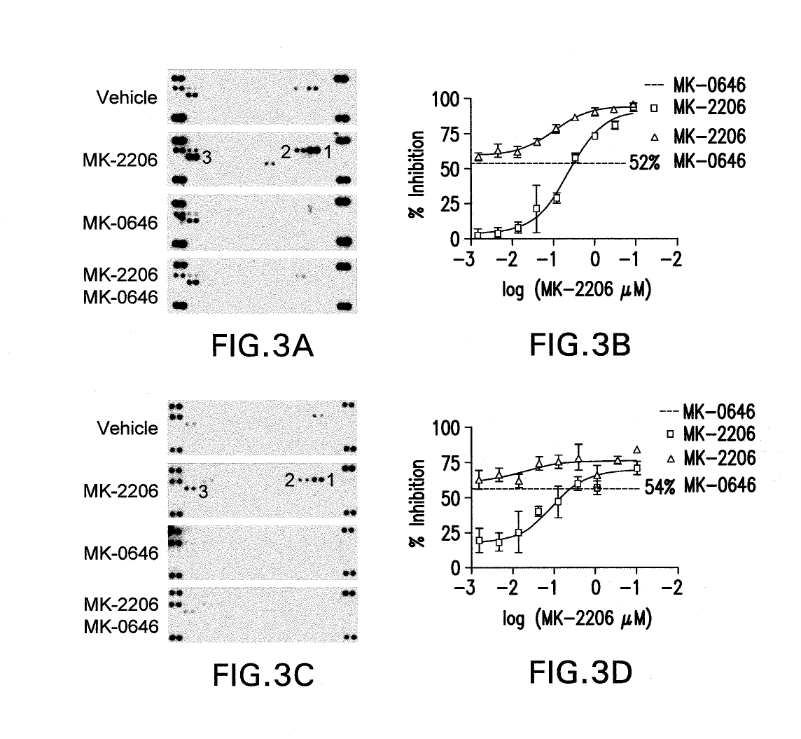

A prediction from the above results would be that the increase in IGF-1R activity after AKT inhibition reduces efficacy of MK-2206. Furthermore, it suggests that inhibition of IGF-1R simultaneously with AKT inhibition may be beneficial. To test this, the two inhibitors were placed on cells either alone or in combination. To evaluate the effects of this combination on IGF-1R activity, phospho-RTK arrays were again utilized. The results show that MK-0646 co-treatment with MK-2206 can negate the upregulation of activity of IGF-1R (FIGS. 3 A and C). Surprisingly, MK-0646 treatment also reduced the amount of active cMET and insulin receptor. These data suggest that MK-0646 may have additional beneficial effects by reducing the activity of other RTKs whose activity is increased upon AKT inhibition.

To study whether the MK-2206/MK-0646 combination would be beneficial functionally in inhibiting cell growth, the combination was assessed in the soft agar colony formation assay. As shown in FIGS. 3 (B and D), the combination of the two inhibitors significantly reduced colony formation compared to the same doses of the single inhibitors. These data together indicate that combining MK-0646 with MK-2206 blocks the MK-2206-mediated upregulation in activity of IGF-1R and provides additional efficacy in vitro.

### In Vivo Efficacy Assessment in Cetuximab Refractory CRC Models

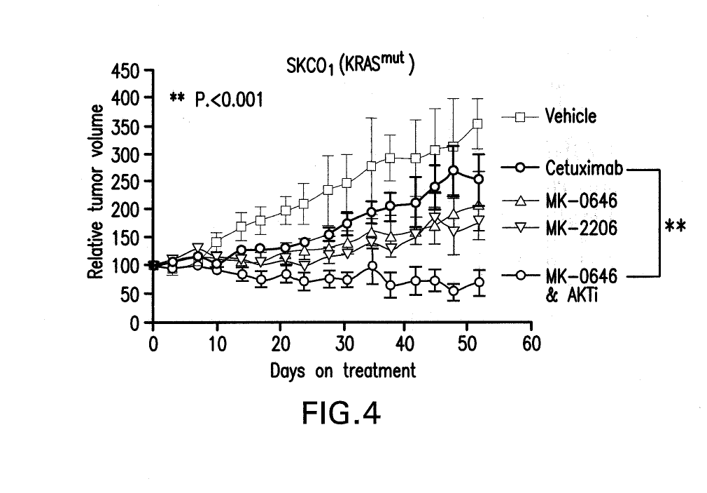

Described herein is data demonstrating the in vivo efficacy of MK-0646 and MK-2206 combination using a cetuximab refractory KRAS mutant CRC xenograft model (SKCO1). SKCO1 tumor bearing mice were treated with either MK-0646 (once a week for 4 weeks), MK-2206 (3-times a week for 4 weeks), or the combination (FIG. 4). MK-0646 (IGF-1R inhibitor—dalotuzumab) and MK-2206 (Akt-Inhibitor) single agent treatments resulted in significant growth inhibition of SKCO1 xenograft tumors, compared to the vehicle treated control group. The single agent treated tumors remained stable in the range of 500-700 mm³ tumor sizes comparable to the start of the treatment. Combined treatment with MK-0646 and MK-2206 significantly enhanced the growth inhibition as compared to single agent treatments (P<0.05) resulting in tumor growth regression (˜20%).

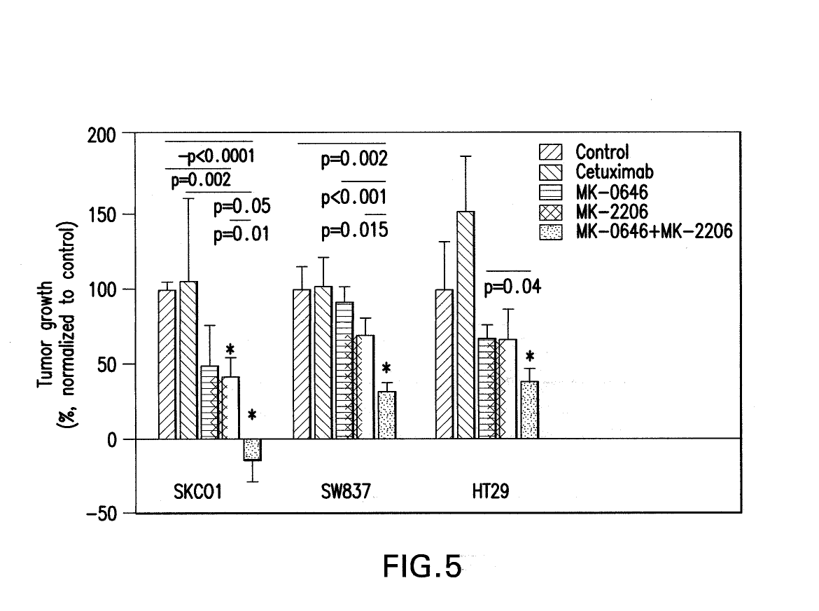

In the cetuximab refractory SW837 model, MK-0646 and MK-2206 single agent treatments resulted in ˜10% and ˜30% tumor growth inhibition, respectively (FIG. 5). However, the MK-0646/MK-2206 combination resulted in statistically significant tumor growth inhibition of ˜60% compared to the vehicle treated group (p=0.002). In this model, the combination also demonstrated statistically significant TGI compared to cetuximab and single agent treatments (all p<0.02). The MK-0646/MK-2206 combination also showed efficacy in one of the most aggressive CRC models—HT29 (FIG. 5). In this model the combination demonstrated significant TGI when compared to cetuximab (p<0.05). The observed combination benefit was not significantly different from MK-0646 and MK-2206 single agent activity.

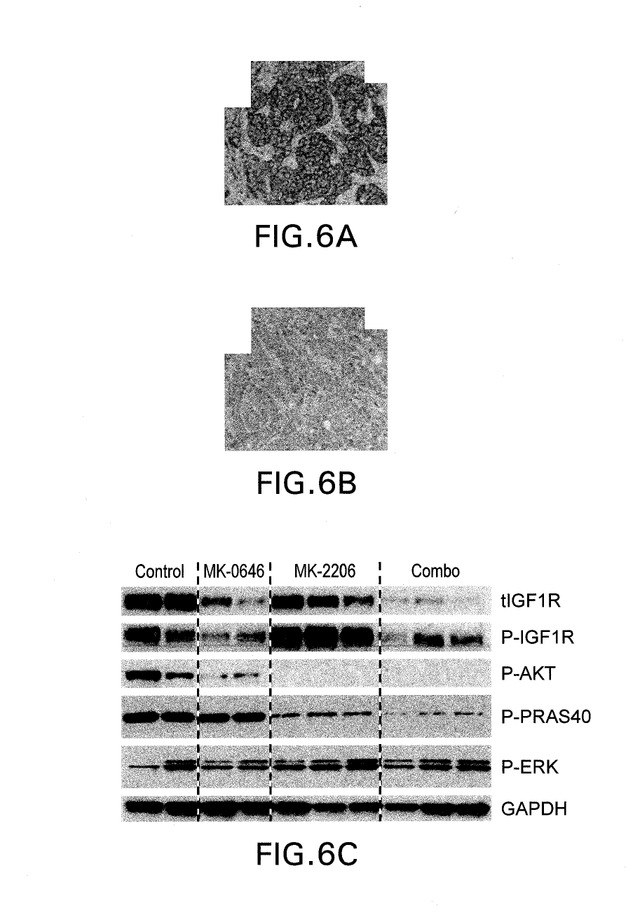

Consistent with the in vitro studies, analysis of the PI3K and IGF-1R signaling components following MK-2206 treatment revealed a feedback activation of IGF-1R signaling as measured by phosphorylation of IGF-1R (FIG. 6). Combined treatment with MK-0646 and MK-2206 blocked this feedback activation and potentiated PI3K pathway inhibition as measured by phosphorylation of 4-EBP1, a key down stream target of mTOR/PI3K signaling regulating protein translation. The enhanced PI3K targeting by the combination is also evident from more profound inhibition of the AKT regulated phosphorylation of PRAS40, a regulator of mTOR activity. These results show the combined treatment with MK-0646 and MK-2206 (AKTi) results in enhanced PI3K pathway inhibition, likely leading to enhanced anti-tumor activity. These studies suggest that MK-0646 in combination with MK-2206 may have utility in the treatment of many tumors in which this feedback activation of IGF-1R by MK-2206 is observed.

### Methods

### Cell Lines and Culture Conditions

All CRC and NSCLC cell lines were obtained from ATCC and maintained in 10% fetal bovine serum, FBS (Hyclone) containing media (DMEM or RPMI; Invitrogen Inc) supplemented with pen-strep (Invitrogen) at 37° C. according to the instructions from ATCC.

### Anchorage Independent Growth Assay

Soft agar assays were conducted in 96 well glass bottom plates (MatriCal), Cells were seeded at a concentration of 3,000-9,000 cells per well in 100 μl RPMI 1640 (Invitrogen) supplemented with 14% FBS and 0.3% (w/v) SeaPlaque Agarose (Lonza Rockland, Inc) on top of a bottom layer of consisting of the same culture media supplemented with 0.8% agarose. Compounds were added in 100 μl of culture media supplemented after agarose had solidified. Cells were incubated for 7-14 days before staining overnight with LavaCell (Active Motif). Colonies were quantified using an Isocyte™ laser scanning cytometer. The ability of MK-0646 to inhibit anchorage independent growth alone or in combination with standard of care agents was evaluated in a soft agar colony forming assay.

### Western Blot Analysis

Total protein lysates from cells (˜0.3 million) cultured in 6 well plates were generated by removing the media, rinsing once with PBS and lysing in NP-40 buffer (1% NP-40, 20 MM Tris-HCL (pH8.0), 137 mM NaCl, 10% glycerol, 2 mM EDTA, Mini Complete protease inhibitor, HALT phosphatase Inhibitor cocktail). Samples were western blotted with indicated total or phosphor-specific antibodies followed by a secondary antibody (Cell Signaling Technology, CST) and then incubated with SuperSignal chemiluminescence substrate (Pierce). The blots were then exposed to a Kodak Biomax Light Film. The antibodies against ERK, p-ERK (Thr202/Tyr204), AKT and p-AKT (Ser473), IGF1R13, S6K & P-S6K (T389), IRS1 & P-IRS1 (S302) and actin were obtained from CST. Total IGF1R antibody was obtained from Santa Cruz and GAPDH was obtained from Chemicon.

### RTK Arrays

About 2 million cells were cultured in 10 cm plates and protein lysates were prepared from a sub-confluent culture. 500 ug of protein lysate were incubated with RTK Arrays (R&D bioscience; cat# ARY001) and developed as detailed in the manual. The arrays were probed with HRP-conjugated P-Tyr antibody and then incubated with Super-Signal chemiluminescence substrate (Pierce) and blots were then exposed to a Kodak Biomax Light Film.

### Xenograft Growth Assessment

A suspension of human SKCO1 and SW837 cells (5×10⁶) and HT29 (3×10⁶) in Matrigel (Cat. #356231, BD Biosciences; 1:1 v/v) were injected subcutaneously into the right flank of 4-6 week old SCID Hairless Outbred (SHO), NOD SCID, or CD-1 nu/nu (Jackson Laboratories) mice, respectively. When tumors reached a size of ˜300 mm3 (Length*Width*Width*0.5), mice were randomized into treatment groups. Mice (n=4-8/group) were dosed with MK-0646 vehicle once per week i.p. for 3 weeks (qwk×3) (20 mM L-Histidine, 150 mM NaCl, 0.5% PS80 pH=6) and MK-2206 vehicle three times per week p.o. for 3 weeks (30% Captisol). The rest of the mice were dosed with either MK-0646 once a week i.p. (20 mg/kg), or MK-2206 three times a week p.o. (140 mg/kg), or the MK-0646/MK-2206 combination using single doses, or cetuximab twice a week i.p. (30 mg/kg). Animals were weighed and tumor volumes were determined by calipering 2 times per week during the study and at termination. Tumor weight was measured and recorded at termination. On day 28 animals were sacrificed by CO₂ asphyxiation. Mice were sacrificed 24 hr after the final MK-0646 dose and 6 hours after the final MK-2206 and combination dose. At time of sacrifice tumors pieces were collected for western blot analysis (snap frozen in liquid nitrogen) and for immunohistochemistry (10% neutral buffered formalin).

### Immunohistochemistry:

Paraffin-embedded tumor sections were analyzed for total IGF1R Ventana Medical Systems), cleaved caspase 3 (Cell Signaling Technologies) and phospho-histone 3 (Millipore Inc.) antibodies. Automated staining was performed using the ChromoMap Kit on the Discovery XT (Ventana Medical Systems) under standard protocol conditions. Images were taken using a Cal Zeiss (Jena, Germany) Imager, Z1 Plan-Apochromat with a 20× objective lens. The images were acquired with a Carl Zeiss AxioCam Hite camera, and the image acquisition software used was Carl Zeiss Axio Vision Rel 4.6. IGF1R was quantified using a qualitative staining index (signal intensity×stained area). Cleaved caspase 3 and phosphor-histone 3 staining was quantified using Gentix ariole system software.

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

What is claimed is:

1. A method of treating a hyper-proliferative disorder in a patient suffering from the disorder comprising administering, to the patient, a combination comprising an isolated antibody or antigen-binding fragment thereof that specifically binds to human Insulin-like growth factor-1 receptor and an Atk inhibitor, wherein administration of the combination results in enhanced therapeutic efficacy in the treatment of the disorder relative to administration of the antibody or antigen-binding fragment alone, wherein the antibody or antigen-binding fragment comprises CDR1, CDR2 and CDR3 of a light chain immunoglobulin comprising an amino acid sequence set forth in SEQ ID NO: 7; and CDR1, CDR2 and CDR3 of a heavy chain immunoglobulin comprising an amino acid sequence set forth in SEQ ID NO: 8.

2. The method of claim 1 where the antibody or antigen-binding fragment thereof comprises a light chain immunoglobulin comprising the amino acid sequence set forth in SEQ ID NO: 7; and a heavy chain immunoglobulin comprising the amino acid sequence set forth in SEQ ID NO: 8.

3. The method of claim 1 wherein the antibody or fragment is an antibody which is dalotuzumab

4. The method according to claim 1, wherein said Akt inhibitor is MK-2206.

5. The method of claim 3 wherein the antibody or fragment is an antibody which is dalotuzumab and said Akt inhibitor is MK-2206.

6. The method of claim 5 wherein the patient is human.

7. The method of claim 1 wherein the disorder is cancer.

8. The method of claim 7 wherein the cancer is:
acute lymphocytic leukemia;
acute nonlymphocytic leukemia;
adrenal cancer;
adult T-cell leukemia/lymphoma;
basal cell carcinoma;
bladder cancer;
bone cancer;
brain cancer;
breast cancer;
chronic lymphocytic leukemia;
chronic myelogenous leukemia;
colon cancer;
colorectal carcinoma;
cutaneous T-cell lymphoma;
endometrium cancer;
esophageal cancer;
Ewing's sarcoma;
genito urinary cancer;
head and neck cancer;
Hodgkin's disease;
Kaposi's sarcoma;
kidney cancer;
laryngeal cancer;
leukemia;
liver cancer;
lung cancer;
lymphoma;
lymphoma associated with human T-cell lymphotrophic virus;
medullary carcinoma;
melanoma;
mesothelioma;
multiple myeloma;
myeloma;
neuroblastoma;
noncutaneous peripheral T-cell lymphoma;
non-Hodgkin's lymphoma;
oral cancer;
osteosarcoma;
ovarian cancer;
pancreatic cancer;
prostate cancer;
rectum cancer;
renal carcinoma;
retinoblastoma;
squamous cell carcinoma;
stomach cancer;
testicular cancer;
thyroid cancer;
uterine cancer;
veticulum cell sarcoma; or
Wilms' tumor.

9. The method of claim 1 wherein the patient is administered a further chemotherapeutic agent.

10. The method of claim 9 wherein the further chemotherapeutic agent is an estrogen receptor modulator, an androgen receptor modulator, a retinoid receptor modulator, a cytotoxic agent, a cytostatic agent, an anti-proliferative agent, a prenyl-protein transferase inhibitor, an HMG-CoA reductase inhibitor, an angiogenesis inhibitor, an HIV protease inhibitor, a reverse transcriptase inhibitor, an inhibitor of cell proliferation and survival signaling, a bisphosphonate, an aromatase inhibitor, an siRNA therapeutic, a γ-secretase inhibitor, an agent that interferes with receptor tyrosine kinases (RTKs) or an agent that interferes with cell cycle checkpoints.

11. A combination comprising:
(i) an isolated antibody or antigen-binding fragment that comprises CDR1, CDR2 and CDR3 of a light chain immunoglobulin comprising an amino acid sequence set forth in SEQ ID NO: 7; and CDR1, CDR2 and CDR3 of a heavy chain immunoglobulin comprising an amino acid sequence set forth in SEQ ID NO: 8; and
(ii) an Atk inhibitor.

12. The combination of claim 11 where the antibody or antigen-binding fragment thereof comprises a light chain immunoglobulin comprising the amino acid sequence set forth in SEQ ID NO: 7; and a heavy chain immunoglobulin comprising the amino acid sequence set forth in SEQ ID NO: 8.

13. The combination of claim 11 wherein the antibody or fragment is an antibody which is dalotuzumab

14. The combination according to claim 11, wherein said Akt inhibitor is MK-2206.

15. The combination of claim 13 wherein the antibody or fragment is an antibody which is dalotuzumab and said Akt inhibitor is MK-2206.

16. The combination of claim 11 further comprising a further chemotherapeutic agent.

17. The combination of claim 16 wherein the further chemotherapeutic agent is an estrogen receptor modulator, an androgen receptor modulator, a retinoid receptor modulator, a cytotoxic agent, a cytostatic agent, an anti-proliferative agent, a prenyl-protein transferase inhibitor, an HMG-CoA reductase inhibitor, an angiogenesis inhibitor, an HIV protease inhibitor, a reverse transcriptase inhibitor, an inhibitor of cell proliferation and survival signaling, a bisphosphonate, an aromatase inhibitor, an siRNA therapeutic, a γ-secretase inhibitor, an agent that interferes with receptor tyrosine kinases (RTKs) or an agent that interferes with cell cycle checkpoints.
